(19) United States
(12) Patent Application Publication
(10) Pub. No.: US 2024/0167046 A1
(43) Pub. Date: May 23, 2024

(54) INDUCIBLE MOSAICISM

(71) Applicant: Syngenta Crop Protection AG, Basel (CH)

(72) Inventors: Esteban Bortiri, Research Triangle Park, NC (US); Timothy Kelliher, Research Triangle Park, NC (US)

(73) Assignee: Syngenta Crop Protection AG, Basel (CH)

(21) Appl. No.: 18/283,741

(22) PCT Filed: Mar. 17, 2022
(86) PCT No.: PCT/US22/20690

§ 371 Date: Sep. 22, 2023

Related U.S. Application Data
(60) Provisional application No. 63/165,224, filed on Mar. 24, 2021.

(51) Int. Cl. C12N 15/82; C07K 14/72; C12N 9/22; C12N 15/10

(52) U.S. Cl. CPC C12N 15/8213; C07K 14/721; C12N 9/22; C12N 15/102; C12N 15/8238; C12N 2310/20

(57) ABSTRACT

Systems and methods for producing a plurality of unique edits in a plant's seed. In one example, a method comprises introducing into a plant cell or a plant tissue a nucleic acid that encodes a DNA modification enzyme; optionally, a nucleic acid that encodes at least one guide RNA (gRNA); and an inducible system sequence, wherein the inducible system sequence is induced at a plant's desired growth stage and either mediates expression of the DNA modification enzyme in at least one of a floral primordia cell and a floral reproductive organ or translocates the DNA modification enzyme to the nucleus; and (ii) mediates a plurality of edits in the at least one of the floral primordia and the floral reproductive organ. The method may also include regenerating the plant cell or plant tissue into a plant having a plurality of seed, wherein the seed contain a plurality of unique edits.

### FIELD OF THE INVENTION

The field of the invention is related to plant biotechnology and specifically to gene editing in plants. The field of the invention further relates to inducible gene editing systems for the purpose of obtaining a plurality of edits in the progeny by inducing the system at a desired stage of the plant life cycle.

### SEQUENCE LISTING

This application is accompanied by a sequence listing entitled INDYMOS_ST25.txt, created Mar. 14, 2022, which is approximately 137 kilobytes in size. This sequence listing is incorporated herein by reference in its entirety. This sequence listing is submitted herewith via EFS-Web and is in compliance with 37 C.F.R. § 1.824(a)(2)-(6) and (b).

### BACKGROUND

The development of scientific methods to improve the quantity and quality of crops is a crucial endeavor. Gene editing, e.g. through targeted mutagenesis, insertion events, allele replacement, etc., is a very important technology widely used to improve both the quantity and quality of various crops. There are numerous methods to edit specific gene targets now, including clustered regularly interspaced short palindromic repeats (CRISPR) and CRISPR-associated sequence (Cas) enzymes, transcription activator-like effector nuclease (TALEN), meganucleases, and zinc fingers. But gene editing is not always an easy task.

Edits to turn off a gene's function (commonly called “knockouts”) can be accomplished relatively easily by genome editing. Through use of a site-directed nuclease, e.g., Cas9 or Cas12a and an associated CRISPR guide RNA (gRNA), one can easily create small insertions or deletions (“indels”) in the coding sequence of a target gene, which frequently lead to frameshifts that truncate the protein or generate an aberrant sequence. In contrast to these well-known methods to knock out a gene, it can be very labor intensive to achieve other types of edits, such as edits that induce a partial loss-of-function or a gain of function allele, or edits that alter the expression level of a gene or the function of the protein product. Many of these edits require allele replacement, which is quite inefficient. Likewise, edits to delete an entire exon or gene or chromosome region (large deletions) can be challenging to execute because they may require simultaneous cutting of more than one gRNA target site. Similarly, edits to introduce a SNP—changing a cytosine nucleotide to a thymine nucleotide, for instance, can utilize base editing technology, but only within certain windows in relation to the targeting site. These are just a few examples of where the desired editing outcome will be challenging to obtain, due to the lack of perfect specificity or efficiency of the DNA modification enzyme system being used.

With respect to allele replacement (sometimes also called “allele swapping”), this is a method of editing that utilizes homologous recombination or homology directed repair, to replace an endogenous sequence in a plant cell with a new sequence that can be provided. While this is reasonably easy to do in yeast and in many animal systems, it is very challenging to do in plants because the non-homologous end joining pathway is strongly favored for DNA repair. Additionally, this process requires delivery of abundant donor DNA to the cut site, to act as the template for DNA repair via homologous recombination. This delivery is not easy to accomplish, particularly for plants. For this reason, allele replacement in plants is typically incredibly expensive and labor intensive to achieve. For example, if one wishes to transform a plant and execute an allele replacement, one may need to generate one thousand stably transformed events to ensure that one allele swap is created in just one or two of the events. The efficiency is generally less than 1%, in some cases, between 0 and 0.3%. Even in the best crops, lines, and construct designs, the efficiency is still very low.

Applicant believes that the cost and labor intensity of generating allele replacements, large deletions, certain base edits, and various other editing outcomes has become a major bottleneck for plant breeding. Few methods have worked to alleviate the extremely low efficiency of the process. Accordingly, the current disclosure is directed to at least one of these, or additional, problems.

Outside of allele replacement, another major challenge for genome editing is the time and labor required to make a wide diversity of sequences (allelic diversity for a locus). For example, it can be quite time consuming and costly to create a diverse array of alleles for a gene's coding sequence, or to create expression diversity by modifying a gene's regulatory region (promoter). The current disclosure is also directed, in many embodiments, to cost-effective methods to produce an allelic series. These and other benefits will become apparent based on the detailed description below.

### SUMMARY

It is the object of this invention to address the challenges around efficiently obtaining heritable edits in a plant. To meet that challenge, one embodiment provides a method for producing a plurality of unique edits in a plant's progeny, comprising: (a) introducing an expression cassette into a plant cell or plant tissue, wherein the expression cassette comprises (i.) a nucleic acid encoding a DNA modification enzyme; (ii.) an optional nucleic acid encoding at least one guide RNA; and (iii.) an inducible factor operably linked to the nucleic acid encoding a DNA modification enzyme; (b.) inducing the inducible factor at a desired plant development stage; and (c.) generating the plant cell or plant tissue into a plant having progeny, wherein the progeny collectively comprise a plurality of unique edits.

In an embodiment, the inducible factor is a transcription effector or a translocation effector; the inducible factor is induced by a chemical, wherein the chemical is selected from an antibiotic, a metal, a steroid, an insecticide, a hormone, an alcohol, and an aldehyde; the antibiotic is tetracycline or a chemical mimic thereof; the metal is copper or a copper-containing compound; the steroid is a glucocorticoid is selected from the group consisting of dexamethasone, beclomethasone, betamethasone, budesonide, cortisone, hydrocortisone, methylprednisolone, prednisolone, prednisone, triamcinolone, and any chemical mimic thereof; the glucocorticoid is dexamethasone; the insecticide is selected from the group consisting of tebufenozide, methoxyfenozide, and any chemical mimic thereof; the hormone is selected from the group consisting of estrogen, oestrogen, 17-β-oestradiol, and any chemical mimic thereof, the alcohol is selected from the group consisting of ethanol and any chemical mimic thereof; the aldehyde is selected from the group consisting of acetaldehyde and any chemical mimic thereof.

In another embodiment, the transcription effector is selected from the group consisting of an alcohol-dependent effector, a lactose-dependent effector, a galactose-dependent effector, and a lexA-dependent effector; the alcohol-dependent effector is an alc effector. In one aspect, the alc effector is an Aspergillus nidulans alc effector comprising an alcA promoter.

In another embodiment, the method further comprises an additional expression cassette comprising a nucleotide sequence encoding an alcR transcription factor activator gene; thereby forming an alcA/alcR inducible system. In one aspect, the method comprises applying an alcohol at the desired plant development stage.

In another embodiment, the lactose-dependent effector is a pOp effector. In one aspect, the method further comprises an additional expression cassette comprising a nucleotide sequence encoding a LhG4 transcription factor activator gene; thereby forming an LhG4/pOp inducible system.

In another embodiment, the galactose-dependent regulon is a Gal4 UAS effector. In one aspect, the method further comprises an additional expression cassette comprising a nucleotide sequence encoding a Gal4 transcription factor activator gene; thereby forming a GVG inducible system or a VGE inducible system.

In another embodiment, the lexA-dependent effector is at least one LexA operon. In one aspect, the method further comprises an additional expression cassette comprising a nucleotide sequence encoding a LexA:VP16:ER activator; thereby forming an XVE inducible system.

In one embodiment, the DNA modification enzyme is selected from the group consisting of a meganuclease (MN), a zinc-finger nuclease (ZFN), a transcription-activator like effector nuclease (TALEN), a chimeric FEN1-FokI, a Mega-TALs, and a CRISPR nuclease. In one aspect, the CRISPR nuclease is a Cas nuclease, a Cas9 nuclease, a Cpf1 nuclease, a dCas9-FokI, a dCpf1-FokI, a chimeric Cas9-cytidine deaminase, a chimeric Cas9-adenine deaminase, a nickase Cas9 (nCas9), a chimeric dCas9 non-FokI nuclease, and a dCpf1 non-FokI nuclease, a Cas12a fused to a deaminase domain, a Cas12i nuclease, a Cas12j nuclease, a CasX nuclease, a CasY nuclease, a Cas13 nuclease, a Cas14 nuclease.

In another embodiment, the translocation factor is a glucocorticoid receptor. In one aspect, the glucocorticoid receptor comprises SEQ ID NO:6. In another aspect, the glucocorticoid receptor is operably linked to a CRISPR nuclease. In another embodiment, the glucocorticoid receptor-linked CRISPR nuclease is a modified Cas12a nuclease modified to comprise a glucocorticoid receptor binding domain (“GR-Cas12a”). In one aspect, the GR-Cas12a comprises SEQ ID NO: 7. In another embodiment, the method further comprises, upon application of dexamethasone, the GR-Cas12a translocates from the cytoplasm to the nucleus of the plant cell or plant tissue.

In another embodiment of the method, the unique edit is an indel mutation, a nucleotide substitution, an allele replacement, a chromosomal translocation, or an insertion of donor nucleic acid.

In another embodiment of the method, the plant cell or plant tissue is dicotyledonous. In one aspect, the dicotyledonous plant cell or plant tissue is selected from the group consisting of Arabidopsis, sunflower, soybean, tomato, Brassica species, Populus (poplar), Eucalyptus, tobacco, Cannabis, potato, cotton, maize, rice, wheat, barley, sugarcane, Glycine tomentella, and other wild Glycine species.

In another embodiment of the method, the plant cell or plant tissue is monocotyledonous. In one aspect, the monocotyledonous plant cell or plant tissue is selected from the group consisting of maize, wheat, rice, teosinte, sorghum, barley. In another aspect, the monocotyledonous plant cell or plant tissue is maize.

In one embodiment, plant cell or plant tissue is maize and wherein the desired developmental stage is selected from the group consisting of VE, V1, V2, V(n), VT, R1, R2, R3, R4, R5, and R6 stage; where (n) is an integer representing the number of leaf collars present.

In another embodiment, plant cell or plant tissue is soybean and wherein the desired developmental stage is selected from the group consisting of VE, VC, V1, V2, V(n), R1, R2, R3, R4, R5, R6, R7, and R8 stage; where (n) is an integer representing the number of trifoliolates present.

In another embodiment, the method further comprises (d.) growing the progeny collectively comprising a plurality of unique edits into seedlings, plantlets, immature plants, mature plants, or senescent plants; (e.) measuring at least one phenotype in the seedlings, plantlets, immature plants, mature plants, or senescent plants of step a.; and (f.) optionally selecting a seedling, plantlet, immature plant, mature plant, or senescent plant based on the measuring of the at least one phenotype.

In yet another embodiment, the method further comprises (d.) growing the progeny collectively comprising a plurality of unique edits into seedlings, plantlets, immature plants, mature plants, or senescent plants; (e.) genotyping the seedlings, plantlets, immature plants, mature plants, or senescent plants of step a.; and (f.) optionally selecting a seedling, plantlet, immature plant, mature plant, or senescent plant based on the genotype of step b.

Another embodiment of the invention is an edited plant produced by the methods recited above.

Another embodiment of the invention is an inducible gene editing system, comprising an expression cassette comprising (a.) a nucleic acid encoding a DNA modification enzyme; (b.) an optional nucleic acid encoding at least one guide RNA; and (c.) an inducible factor operably linked to the nucleic acid encoding a DNA modification enzyme. In one embodiment, the system further comprises a cell harboring the expression cassette. In one aspect, the cell is a eukaryotic cell. In another aspect, the eukaryotic cell is a plant cell.

### BRIEF DESCRIPTION OF THE SEQUENCES IN THE SEQUENCE LISTING

SEQ ID NO: 1 is vector 24902. It comprises the nucleotide sequence for a constitutively expressed GVG protein. See also FIG. 1.

SEQ ID NO: 2 is vector 25657. It comprises the nucleotide sequence for rice codon-optimized GR-LbCas12, which lacks a nuclear localization signal (“NLS”) and has a glucocorticoid receptor (“GR”) binding domain at the N-terminus separated by a long linker. The resulting chimeric GR-Cas12 is constitutively expressed but localized to the cytoplasm. While in the presence of DEX, GR-Cas12a translocates to the nucleus. See also FIG. 2.

SEQ ID NO: 3 is vector 25765. It comprises the nucleotide sequence for the AlcA/AlcR ethanol-dependent inducible system. See also FIG. 3.

SEQ ID NO: 4 is vector 25881. It comprises the nucleotide sequence for the AlcA/AlcR ethanol-dependent inducible system to induce expression of Cas12a when in the presence of ethanol and/or acetaldehyde. See also FIG. 4.

SEQ ID NO: 5 is an amino acid for a GVG protein.

SEQ ID NO: 6 is an amino acid sequence for a glucocorticoid receptor.

SEQ ID NO: 7 is an amino acid sequence for a Cas12a protein having a fused glucocorticoid receptor.

SEQ ID NO: 8 is the 614 base pair gl1 fragment amplicon.

SEQ ID NO: 9 is the primer GL1_F used to produce the gl1 amplicon.

SEQ ID NO: 10 is the primer GL1_R used to produce the gl1 amplicon.

SEQ ID NO: 11 is an example gl1 consensus sequence.

SEQ ID NO: 12 is an edit of the gl1 sequence.

SEQ ID NO: 13 is an edit of the gl1 sequence.

SEQ ID NO: 14 is an edit of the gl1 sequence.

SEQ ID NO: 15 is an edit of the gl1 sequence.

SEQ ID NO: 16 is an edit of the gl1 sequence.

SEQ ID NO: 17 is an edit of the gl1 sequence.

SEQ ID NO: 18 is an edit of the gl1 sequence.

SEQ ID NO: 19 is an edit of the gl1 sequence.

SEQ ID NO: 20 is an edit of the gl1 sequence.

SEQ ID NO: 21 is an edit of the gl1 sequence.

SEQ ID NO: 22 is an edit of the gl1 sequence.

SEQ ID NO: 23 is an edit of the gl1 sequence.

SEQ ID NO: 24 is an edit of the gl1 sequence.

SEQ ID NO: 25 is an edit of the gl1 sequence.

SEQ ID NO: 26 is an edit of the gl1 sequence.

SEQ ID NO: 27 is an edit of the gl1 sequence.

SEQ ID NO: 28 is an edit of the gl1 sequence.

SEQ ID NO: 29 is an edit of the gl1 sequence.

SEQ ID NO: 30 is an edit of the gl1 sequence.

SEQ ID NO: 31 is an edit of the gl1 sequence.

SEQ ID NO: 32 is an edit of the gl1 sequence.

SEQ ID NO: 33 is an edit of the gl1 sequence.

SEQ ID NO: 34 is an edit of the gl1 sequence.

SEQ ID NO: 35 is an edit of the gl1 sequence.

SEQ ID NO: 36 is an edit of the gl1 sequence.

SEQ ID NO: 37 is an edit of the gl1 sequence.

SEQ ID NO: 38 is an edit of the gl1 sequence.

SEQ ID NO: 39 is an edit of the gl1 sequence.

SEQ ID NO: 40 is an edit of the gl1 sequence.

SEQ ID NO: 41 is an edit of the gl1 sequence.

SEQ ID NO: 42 is an edit of the gl1 sequence.

SEQ ID NO: 43 is an edit of the gl1 sequence.

SEQ ID NO: 44 is an edit of the gl1 sequence.

SEQ ID NO: 45 is an edit of the gl1 sequence.

SEQ ID NO: 46 is an edit of the gl1 sequence.

SEQ ID NO: 47 is an edit of the gl1 sequence.

SEQ ID NO: 48 is an edit of the gl1 sequence.

SEQ ID NO: 49 is an edit of the gl1 sequence.

SEQ ID NO: 50 is an edit of the gl1 sequence.

SEQ ID NO: 51 is an edit of the gl1 sequence.

SEQ ID NO: 52 is an edit of the gl1 sequence.

SEQ ID NO: 53 is an edit of the gl1 sequence.

SEQ ID NO: 54 is an edit of the gl1 sequence.

SEQ ID NO: 55 is an edit of the gl1 sequence.

SEQ ID NO: 56 is an edit of the gl1 sequence.

SEQ ID NO: 57 is an edit of the gl1 sequence.

SEQ ID NO: 58 is an edit of the gl1 sequence.

SEQ ID NO: 59 is an edit of the gl1 sequence.

SEQ ID NO: 60 is an edit of the gl1 sequence.

SEQ ID NO: 61 is an edit of the gl1 sequence.

SEQ ID NO: 62 is an edit of the gl1 sequence.

SEQ ID NO: 63 is an edit of the gl1 sequence.

SEQ ID NO: 64 is an edit of the gl1 sequence.

SEQ ID NO: 65 is an edit of the gl1 sequence.

SEQ ID NO: 66 is an edit of the gl1 sequence.

SEQ ID NO: 67 is an edit of the gl1 sequence.

SEQ ID NO: 68 is an edit of the gl1 sequence.

SEQ ID NO: 69 is vector 27057. It comprises the nucleotide sequence for the dexamethasone-inducible expression of LbCas12a. cGa14VP16GR is constitutively expressed and, in the presence of DEX, it localizes to the nucleus, binds to the GAL4 UAS promoter, and drives transcription of LbCas12a. The guide RNA targets the second exon of the Glabrous 1(GL1) gene for phenotypic screening. The construct contains Kanamycin resistance cassette for selection of Arabidopsis transformants. The guide RNA is expressed using the ribozyme hammerhead design from a soybean S-adenosylmethionine synthetase (SAMS) promoter. See also FIG. 6.

### BRIEF DESCRIPTION OF THE FIGURES

FIG. 1 is a plasmid map for vector 24902.

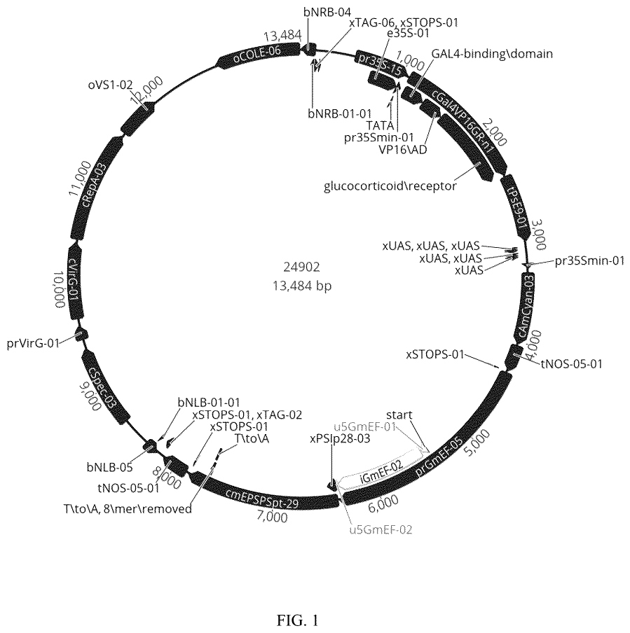

FIG. 2 is a plasmid map for vector 25657.

FIG. 3 is a plasmid map for vector 25765.

FIG. 4 is a plasmid map for vector 25881.

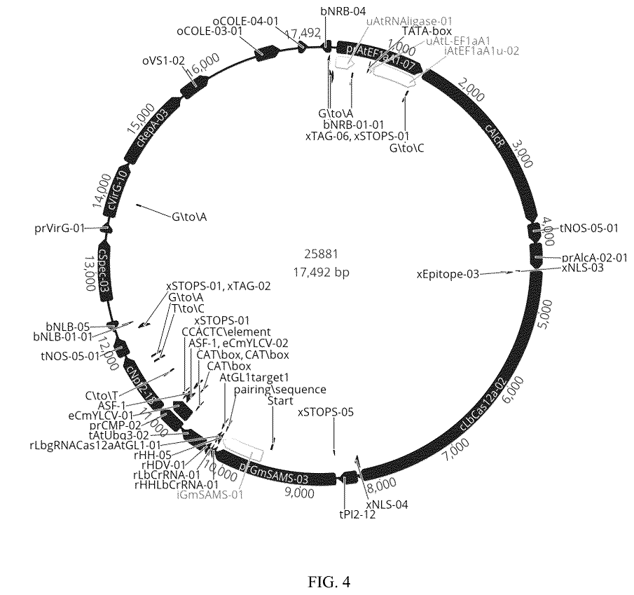

FIG. 5 is a photograph of the appearance of the wildtype and the gl1 mutant in Arabidopsis.

FIG. 6 is a plasmid map for vector 27057.

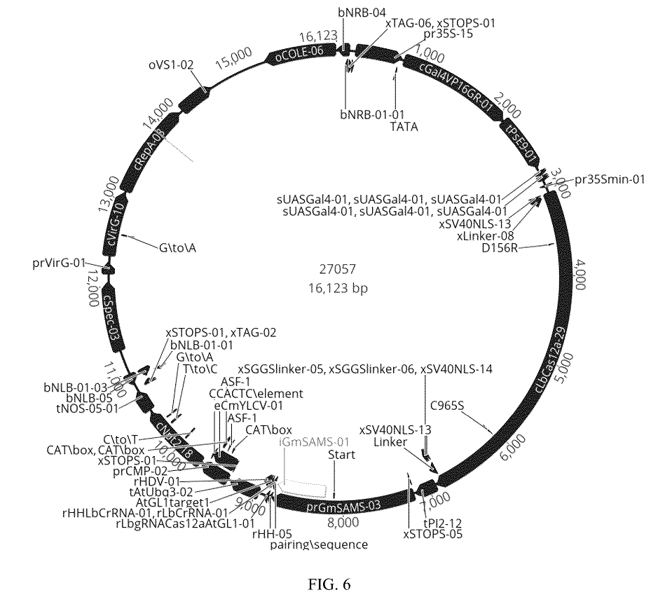

### DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

As used herein, a “CRISPR enzyme” means any Type I, II, IV, or V enzyme isolated from a bacterial CRISPR system or any artificial, synthetic, or otherwise altered homolog thereof. In particular, this definition encompasses Cas9, Cas12a (also known as Cpf1), Cas12i, Cas12j, Cms1, MAD7, Cas13, Cas14, and the like, and mutants thereof. See U.S. Pat. Nos. 10,227,611; 10,000,772; 9,790,490; 9,896,696; 9,982,279; WO2014/093595; WO2017/184768; WO2018/195545; all of which are incorporated herein by reference in their entirety. Additionally, modifications of these enzymes are within the scope of this definition, for example, a fusion enzyme comprising a deaminase domain, or an exonuclease domain, a transposase domain, a reverse-transcriptase domain, and the like, e.g., Cas9-BE (a fusion of Cas9 and a base editor domain, e.g., APOBEC; see), Cas12a-BE (a fusion of Cas12a and a base editor domain, e.g., APOBEC, and further optionally comprising a uracil DNA glycosylase), or Cas9-RT (a Cas enzyme fused to a reverse transcriptase domain; see WO2020/191233 incorporated herein by reference in its entirety). Likewise, nuclease-inactive (“dCas”) or nickase (“nCas”) versions of these enzymes are within the scope of this definition. “CRISPR enzyme” and “CRISPR nuclease” are used interchangeably throughout.

As used herein, “inducible mosaicism” refers to the use of an inducible system to obtain a mosaicism of edits in progeny plant. Applicable inducible systems include but are not limited to an AlcA/AlcR inducible system, an LhG4/pOp inducible system, a GVG inducible system, and a VGE inducible system. An inducible system is tethered, functionally, operably, or physically to a CRISPR enzyme. Upon induction of the inducible system, the CRISPR enzyme is expressed or alternatively translocated to the nucleus. To obtain mosaicism in plants, it is important that the induction occurs in coincidence with the development of the plant tissue of interest. If mosaicism is desired at the development of a leaf, the induction will occur at approximately when the leaf cells begin to develop and/or differentiate. If mosaicism is desired in the progeny of a plant, the induction will occur at approximately when the floral primordia cells begin to development.

As used herein, “chemical mimic” means a chemical having a similar structure and/or effect as another chemical. For example, a chemical mimic of dexamethasone may share a similar structure as dexamethasone, or it may be a modified version of dexamethasone. In either instance, the chemical mimic of dexamethasone will be capable of performing a similar function as dexamethasone in a DEX-inducible system. Additionally, a chemical mimic of acetaldehyde may share a similar structure as acetaldehyde, or it may be a modified version of acetaldehyde. In either instance, the chemical mimic of acetaldehyde will be capable of performing a similar function as acetaldehyde in an AlcA/AlcR-inducible system. Likewise, a chemical mimic of ethanol can be metabolized into acetaldehyde, similar to ethanol's metabolism into acetaldehyde, in order to function in an AlcA/AlcR-inducible system.

As used herein, “genotyping” refers to any analytical method of analyzing an organism's or cell's genetic code. Methods of genotyping include, among others, Sanger sequencing, next-generation sequencing (“NGS”), polymerase chain reaction (“PCR”), and TaqMan analysis. Genotyping may include PCR amplification of the target region followed by Sanger sequencing and deconvolution of chromatograms using ICE analysis (see ice.synthego.com). Genotyping methods may be manual or automated. Genotyping includes whole genome sequencing, SNP detection, haplotype analysis, zygosity analysis, and adventitious presence analysis.

As used herein, “translocation effector” refers to a molecule (proteinaceous or otherwise) upon which movement within a cell is dependent. For example, and not by way of limitation, a glucocorticoid receptor operates as a translocation effector when fused to a heterologous protein.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

As used herein, the word “and/or” refers to and encompasses any and all possible combinations of one or more of the associated listed items, as well as the lack of combinations when interpreted in the alternative, “or” and refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter. Where the term “about” is used in the context of this disclosure (e.g., in combinations with temperature or molecular weight values) the exact value (i.e., without “about”) can be preferred.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

Units, prefixes and symbols may be denoted in their SI accepted form. Unless otherwise indicated, nucleic acids are written left to right in 5′ to 3′ orientation; amino acid sequences are written left to right in N-terminus to C-terminus orientation, respectively. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the phrase “associated with” refers to a recognizable and/or assayable relationship between two entities. For example, the phrase “associated with HI” refers to a trait, locus, gene, allele, marker, phenotype, etc., or the expression thereof, the presence or absence of which can influence an extent and/or degree at which a plant or its progeny exhibits HI or haploid induction. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with HI” refers to a marker whose presence or absence can be used to predict whether and/or to what extent a plant will display haploid induction.

“Associated with/operatively linked” can also refer to two nucleic acids that are related physically or functionally. For example, a promoter or regulatory DNA sequence is said to be “associated with” a DNA sequence that codes for RNA or a protein if the two sequences are operatively linked, or situated such that the regulatory DNA sequence will affect the expression level of the coding or structural DNA sequence.

A “coding sequence” is a nucleic acid sequence that is transcribed into RNA such as mRNA, rRNA, tRNA, snRNA, sense RNA or antisense RNA which is then preferably translated in an organism to produce a protein.

As used herein, a “codon optimized” sequence means a nucleotide sequence wherein the codons are chosen to reflect the particular codon bias that a host cell or organism may have. This is typically done in such a way so as to preserve the amino acid sequence of the polypeptide encoded by the nucleotide sequence to be optimized. In certain embodiments, the DNA sequence of the recombinant DNA construct includes sequence that has been codon optimized for the cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, which is incorporated herein by reference. In embodiments, the polynucleotides of the disclosure are codon-optimized for expression in a plant cell (e.g., a dicot cell or a monocot cell) or bacterial cell.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

As used herein, the phrase “consisting essentially of” (and grammatical variants) limits the scope of the related disclosure or claim to the specified materials and/or steps, plus those that do not materially affect the basic and novel characteristic(s) of the disclosed and/or claimed subject matter. The terms “comprises”, “comprising, “includes”, “including”, “having” and their conjugates mean including “but not limited to”. These terms specify the presence of stated features, integers, steps, operations, elements, or components, but do not preclude the presence or addition of one or more other features, integers, steps, operations, elements, components, or groups thereof. The term “consisting of means “including and limited to”.

With respect to the terms “comprising,” “consisting essentially of,” and “consisting of,” where one of these three terms is used herein, the presently disclosed and claimed subject matter can include in some embodiments the use of either of the other two terms. For example, if a subject matter relates in some embodiments to nucleic acids that encode polypeptides comprising amino acid sequences that are at least 95% identical to a SEQ ID NO:. It is understood that the disclosed subject matter thus also encompasses nucleic acids that encode polypeptides that in some embodiments consist essentially of amino acid sequences that are at least 95% identical to that SEQ ID NO: as well as nucleic acids that encode polypeptides that in some embodiments consist of amino acid sequences that are at least 95% identical to that SEQ ID NO. Similarly, it is also understood that in some embodiments the methods for the disclosed subject matter comprise the steps that are disclosed herein, in some embodiments the methods for the presently disclosed subject matter consist essentially of the steps that are disclosed, and in some embodiments the methods for the presently disclosed subject matter consist of the steps that are disclosed herein.

In the context of the disclosure, “corresponding to” or “corresponds to” means that when the amino acid sequences of a reference sequence are aligned with a second amino acid sequence (e.g. variant or homologous sequences), different from the reference sequence, the amino acids that “correspond to” certain enumerated positions in the second amino acid sequence are those that align with these positions in the reference amino acid sequence but that are not necessarily in the exact numerical positions relative to the particular reference amino acid sequence of the disclosure.

As used herein, the term “event” refers to a genetically engineered organism or cell, for example, a genetically engineered plant or seed made to have non-natural DNA, which would not normally be found in nature. Events may include transgenic events where a transgene is been inserted into the DNA of an organism. Events may also include the insertion of a particular transgene into a specific location on a chromosome. Events may also include any combination of indels and point mutations.

As used herein, the term “gene” refers to a hereditary unit including a sequence of DNA that occupies a specific location on a chromosome and that contains the genetic instruction for a particular characteristic or trait in an organism.

A “genetic map” is a description of genetic linkage relationships among loci on one or more chromosomes within a given species, generally depicted in a diagrammatic or tabular form.

As used herein a “gene regulatory network” (or “GRN”) is a collection of molecular regulators that interact with each other and with other substances in the cell to govern the gene expression levels of mRNA and proteins. The regulator can be DNA, RNA, protein and complexes of these. GRNs may also be inclusive of a “gene family” as used herein. A “gene family” refers to a set of several similar genes, with generally similar biochemical functions.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide group.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may have at least one of its components heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

An expression cassette comprising a nucleotide sequence of interest may be chimeric, meaning that at least one of its components is heterologous with respect to at least one of its other components. An expression cassette may also be one that comprises a native promoter driving its native gene; however, it has been obtained in a recombinant form useful for heterologous expression. Such usage of an expression cassette makes it so it is not naturally occurring in the cell into which it has been introduced.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used. Any available terminator known to function in plants can be used in the context of this disclosure.

The term “heterologous” when used in reference to a gene or a polynucleotide or a polypeptide refers to a gene or a polynucleotide or a polypeptide that is or contains a part thereof not in its natural environment (i.e., has been altered by the hand of man). For example, a heterologous gene may include a polynucleotide from one species introduced into another species. A heterologous gene may also include a polynucleotide native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the disclosure, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, a “heterologous” polynucleotide refers to a polynucleotide not naturally associated with a host cell into which it is introduced, including non-naturally occurring multiple copies of a naturally occurring polynucleotide. A heterologous nucleic acid sequence or nucleic acid molecule may comprise a chimeric sequence such as a chimeric expression cassette, where the promoter and the coding region are derived from multiple source organisms. The promoter sequence may be a constitutive promoter sequence, a tissue-specific promoter sequence, a chemically-inducible promoter sequence, a wound-inducible promoter sequence, a stress-inducible promoter sequence, or a developmental stage-specific promoter sequence.

A “homologous” nucleic acid sequence is a nucleic acid sequence naturally associated with a host cell into which it is introduced.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

As used herein, a plant referred to as “haploid” has a reduced number of chromosomes (n) in the haploid plant, and its chromosome set is equal to that of the gamete. In a haploid organism, only half of the normal number of chromosomes are present. Thus haploids of diploid organisms (e.g., maize) exhibit monoploidy; haploids of tetraploid organisms (e.g., ryegrasses) exhibit diploidy; haploids of hexaploid organisms (e.g., wheat) exhibit triploidy; etc. As used herein, a plant referred to as “doubled haploid” is developed by doubling the haploid set of chromosomes. A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric in vegetative tissues.

As used herein, the term “human-induced mutation” refers to any mutation that occurs as a result of either direct or indirect human action. This term includes, but is not limited to, mutations obtained by any method of targeted mutagenesis.

As used herein, “introduced” means delivered, expressed, applied, transported, transferred, permeated, or other like term to indicate the delivery, whether of nucleic acid or protein or combination thereof, of a desired object to an object. For example, nucleic acids encoding a site directed nuclease and optionally at least one guide RNA may be introduced into a plant cell.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

As used herein, the term “molecular marker” can be used to refer to a genetic marker, as defined above, or an encoded product thereof (e.g., a protein) used as a point of reference when identifying the presence/absence of a HI-associated locus. A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the term “nucleotide sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100.

One example of an algorithm that is suitable for determining percent sequence identity and sequence similarity is the BLAST algorithm, which is described in Altschul et al., 1990. In some embodiments, a percentage of sequence identity refers to sequence identity over the full length of one of the gDNA, cDNA, or the predicted protein sequences in the largest ORF of SEQ ID No: 1 being compared. In some embodiments, a calculation to determine a percentage of nucleic acid sequence identity does not include in the calculation any nucleotide positions in which either of the compared nucleic acids includes an “N” (i.e., where any nucleotide could be present at that position).

The term “open reading frame” (ORF) refers to a nucleic acid sequence that encodes a polypeptide. In some embodiments, an ORF comprises a translation initiation codon (i.e., start codon), a translation termination (i.e., stop codon), and the nucleic acid sequence there between that encodes the amino acids present in the polypeptide. The terms “initiation codon” and “termination codon” refer to a unit of three adjacent nucleotides (i.e., a codon) in a coding sequence that specifies initiation and chain termination, respectively, of protein synthesis (mRNA translation).

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In other cases, a phenotype is the result of interactions among several genes, which in some embodiments also results from an interaction of the plant and/or plant cell with its environment.

As used herein, the term “plant” can refer to a whole plant, any part thereof, or a cell or tissue culture derived from a plant. The class of plants, which can be used in the methods of the disclosure, is generally as broad as the class of higher plants amenable to transformation techniques, including both monocotyledonous and dicotyledonous plants including species from the genera but not limited to: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Maize, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the terms “progeny” and “progeny plant” refer to a plant generated from vegetative or sexual reproduction from one or more parent plants. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1s, F2s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids.

A “portion” or a “fragment” of a polypeptide of the disclosure will be understood to mean an amino acid sequence or nucleic acid sequence of reduced length relative to a reference amino acid sequence or nucleic acid sequence of the disclosure. Such a portion or a fragment according to the disclosure may be, where appropriate, included in a larger polypeptide or nucleic acid of which it is a constituent (e.g., a tagged or fusion protein or an expression cassette). In embodiments, the “portion” or “fragment” substantially retains the activity, such as insecticidal activity (e.g., at least 40%, 50%, 60%, 70%, 80%, 85%, 90%, 95% or even 100% of the activity) of the full-length protein or nucleic acid, or has even greater activity, e.g., insecticidal activity, than the full-length protein).

The terms “protein,” “peptide,” and “polypeptide” are used interchangeably herein.

The term “promoter,” as used herein, refers to a polynucleotide, usually upstream (5′) of the translation start site of a coding sequence, which controls the expression of the coding sequence by providing the recognition for RNA polymerase and other factors required for proper transcription. For example, a promoter may contain a region containing basal promoter elements recognized by RNA polymerase, a region containing the 5′ untranslated region (UTR) of a coding sequence, and optionally an intron.

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer in some embodiments to a meiotic crossover.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule which may be incorporated into its genome. As a result of such genomic alteration, the recombinant plant is distinctly different from the related wild-type plant. A “recombinant” bacteria is a bacteria not found in nature that comprises a heterologous nucleic acid molecule. Such a bacteria may be created by transforming the bacteria with the nucleic acid molecule or by the conjugation-like transfer of a plasmid from one bacteria strain to another, whereby the plasmid comprises the nucleic acid molecule.

As used herein, the term “reference sequence” refers to a defined nucleotide sequence used as a basis for nucleotide sequence comparison.

As used herein, the term “regenerate,” and grammatical variants thereof, refers to the production of a plant from tissue culture.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translational enhancer sequences, introns, terminators, and polyadenylation signal sequences. They include natural and synthetic sequences as well as sequences which may be a combination of synthetic and natural sequences. Regulatory sequences may determine expression level, the spatial and temporal pattern of expression and, for a subset of promoters, expression under inductive conditions (regulation by external factors such as light, temperature, chemicals and hormones).

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances.

Longer sequences typically hybridize specifically at higher temperatures. An extensive guide to the hybridization of nucleic acids is found in Sambrook & Russell, 2001. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Exemplary stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides).

Stringent conditions can also be achieved with the addition of destabilizing agents such as formamide. Additional exemplary stringent hybridization conditions include 50% formamide, 5×SSC, and 1% SDS incubating at 42° C.; or SSC, 1% SDS, incubating at 65° C.; with one or more washes in 0.2×SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al., 1999).

As used herein, the term “trait” refers to a phenotype of interest, a gene that contributes to a phenotype of interest, as well as a nucleic acid sequence associated with a gene that contributes to a phenotype of interest. For example, a “HI trait” refers to a haploid induction phenotype as well as a gene (e.g., matl in maize or Os03g27610 in rice) that contributes to a haploid induction and a nucleic acid sequence (e.g., a HI-associated gene product) that is associated with the presence or absence of the haploid induction phenotype.

As used herein, the term “transgene” refers to a nucleic acid molecule introduced into an organism or one or more of its ancestors by some form of artificial transfer technique. The artificial transfer technique thus creates a “transgenic organism” or a “transgenic cell.” It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

“Transformation” is a process for introducing heterologous nucleic acid into a host cell or organism. In particular embodiments, “transformation” means the stable integration of a DNA molecule into the genome (nuclear or plastid) of an organism of interest. In some particular embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or a combination thereof. Procedures for transforming plants are well known and routine in the art and are described throughout the literature.

Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

“Transformed” and “transgenic” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

It is specifically contemplated that one could mutagenize a promoter to potentially improve the utility of the elements for the expression of transgenes in plants. The mutagenesis of these elements can be carried out at random and the mutagenized promoter sequences screened for activity in a trial-by-error procedure. Alternatively, particular sequences which provide the promoter with desirable expression characteristics, or the promoter with expression enhancement activity, could be identified and these or similar sequences introduced into the promoter via mutation. It is further contemplated that one could mutagenize these sequences in order to enhance their expression of transgenes in a particular species. The means for mutagenizing a DNA segment encoding a promoter sequence of the current invention are well-known to those of skill in the art. As indicated, modifications to promoter or other regulatory element may be made by random, or site-specific mutagenesis procedures. The promoter and other regulatory element may be modified by altering their structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory sequence. In particular, site-specific mutagenesis is a technique useful in the preparation of promoter mutants, through specific mutagenesis of the underlying DNA. RNA-guided endonucleases (“RGEN,” e.g., CRISPR/Cas9) may also be used. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

Where a clone comprising a promoter has been isolated in accordance with the instant invention, one may wish to delimit the promoter regions within the clone. One efficient, targeted means for preparing mutagenized promoters relies upon the identification of putative regulatory elements within the promoter sequence. This can be initiated by comparison with promoter sequences known to be expressed in similar tissue specific or developmentally unique patterns. Sequences which are shared among promoters with similar expression patterns are likely candidates for the binding of transcription factors and are thus likely elements which confer expression patterns. Confirmation of these putative regulatory elements can be achieved by deletion analysis of each putative regulatory sequence followed by functional analysis of each deletion construct by assay of a reporter gene which is functionally attached to each construct. As such, once a starting promoter sequence is provided, any of a number of different deletion mutants of the starting promoter could be readily prepared.

The invention disclosed herein provides polynucleotide molecules comprising regulatory element fragments that may be used in constructing novel chimeric regulatory elements. Novel combinations comprising fragments of these polynucleotide molecules and at least one other regulatory element or fragment can be constructed and tested in plants and are considered to be within the scope of this invention. Thus the design, construction, and use of chimeric regulatory elements is one embodiment of this invention. Promoters of the present invention include homologues of cis elements known to affect gene regulation that show homology with the promoter sequences of the present invention.

Functional equivalent fragments of one of the transcription regulating nucleic acids described herein comprise at least 50, 100, 150, 200, 250, 300, 350, 400, 450, 500, 550, 600, 650, 700, 750, 800, 850, 900, 950, or 1000 base pairs of a transcription regulating nucleic acid. Equivalent fragments of transcription regulating nucleic acids, which are obtained by deleting the region encoding the 5′-untranslated region of the mRNA, would then only provide the (untranscribed) promoter region. The 5′-untranslated region can be easily determined by methods known in the art (such as 5′-RACE analysis). Accordingly, some of the transcription regulating nucleic acids, described herein, are equivalent fragments of other sequences.

As indicated above, deletion mutants of the promoter of the invention also could be randomly prepared and then assayed. Following this strategy, a series of constructs are prepared, each containing a different portion of the promoter (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

“At least one expression cassette” as described herein refers to, inter alia, DNA including an inducible system sequence and a nucleic acid that encodes a DNA modification enzyme to be expressed by a cell. In one example, the at least one expression cassette is a component of a vector DNA and is expressed after transformation in a cell. The at least one expression cassette as described herein will often include multiple expression cassettes, for example: an expression cassette comprising a regulatory sequence and a nucleic acid encoding a gRNA; an expression cassette comprising a regulatory sequence initiating replication of a Donor DNA; an expression cassette comprising a regulatory sequence and a selectable marker, or some combination thereof, for example an expression cassette comprising DNA encoding a Cas enzyme and a gRNA under the control of an inducible system sequence. The at least one expression cassette as described herein may comprise further regulatory elements. The term in this context is to be understood in the broad meaning comprising all sequences which may influence construction or function of the at least one expression cassette. Regulatory elements may, for example, modify transcription and/or translation in prokaryotic or eukaryotic organisms. The at least one expression cassette described herein may be downstream (in 3′ direction) of the nucleic acid sequence to be expressed and optionally contain additional regulatory elements, such as transcriptional or translational enhancers. Each additional regulatory element may be operably liked to the nucleic acid sequence to be expressed (or the transcription regulating nucleotide sequence). Additional regulatory elements may comprise additional promoters, minimal promoters, promoter elements, or transposon elements which may modify or enhance the expression regulating properties. The at least one expression cassette may also contain one or more introns, one or more exons and one or more terminators.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein, e.g. FMOS promoters, may be combined with elements from other promoters, FMOS or otherwise, so long as FMOS function is maintained. For example, in certain embodiments introns in FMOS promoters may be replaced with introns from other promoters, such as, an intron from a ubiquitin promoter. Further still FMOS promoters may be lengthened in some embodiments, e.g., by fusing with introns from other promoters, such as, for example, fusing an FMOS promoter with an intron from a ubiquitin promoter.

The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Example vectors include a plasmid, cosmid, phagemid, artificial chromosome, phage or viral vector.

### DETAILED DESCRIPTION

The disclosure is directed to, inter alia, systems and methods to improve gene editing efficiencies, for example, to reduce the number of transformations required to generate edits, e.g., new mutations or events in a plant's DNA.

In various embodiments, the disclosure is directed to methods for producing a plurality of unique edits in a plant's seed, e.g. a plurality of unique allele replacements, a plurality of unique base insertions, a plurality of unique base deletions, or a plurality of unique point mutations.

One embodiment provides a method for producing a plurality of unique edits in a plant's progeny, comprising: (a) introducing an expression cassette into a plant cell or plant tissue, wherein the expression cassette comprises (i.) a nucleic acid encoding a DNA modification enzyme; (ii.) an optional nucleic acid encoding at least one guide RNA; and (iii.) an inducible factor operably linked to the nucleic acid encoding a DNA modification enzyme; (b.) inducing the inducible factor at a desired plant development stage; and (c.) generating the plant cell or plant tissue into a plant having progeny, wherein the progeny collectively comprise a plurality of unique edits. In an embodiment, the inducible factor is a transcription effector or a translocation effector; the inducible factor is induced by a chemical, wherein the chemical is selected from an antibiotic, a metal, a steroid, an insecticide, a hormone, an alcohol, and an aldehyde; the antibiotic is tetracycline or a chemical mimic thereof, the metal is copper or a copper-containing compound; the steroid is a glucocorticoid is selected from the group consisting of dexamethasone, beclomethasone, betamethasone, budesonide, cortisone, hydrocortisone, methylprednisolone, prednisolone, prednisone, triamcinolone, and any chemical mimic thereof, the glucocorticoid is dexamethasone; the insecticide is selected from the group consisting of tebufenozide, methoxyfenozide, and any chemical mimic thereof, the hormone is selected from the group consisting of estrogen, oestrogen, 17-β-oestradiol, and any chemical mimic thereof; the alcohol is selected from the group consisting of ethanol and any chemical mimic thereof; the aldehyde is selected from the group consisting of acetaldehyde and any chemical mimic thereof. In another embodiment, the transcription effector is selected from the group consisting of an alcohol-dependent effector, a lactose-dependent effector, a galactose-dependent effector, and a lexA-dependent effector; the alcohol-dependent effector is an alc effector. In one aspect, the alc effector is an Aspergillus nidulans alc effector comprising an alcA promoter.

In another embodiment, the method further comprises an additional expression cassette comprising a nucleotide sequence encoding an alcR transcription factor activator gene; thereby forming an alcA/alcR inducible system. In one aspect, the method comprises applying an alcohol at the desired plant development stage.

In another embodiment, the lactose-dependent effector is a pOp effector. In one aspect, the method further comprises an additional expression cassette comprising a nucleotide sequence encoding a LhG4 transcription factor activator gene; thereby forming an LhG4/pOp inducible system.

In another embodiment, the galactose-dependent regulon is a Gal4 UAS effector. In one aspect, the method further comprises an additional expression cassette comprising a nucleotide sequence encoding a Gal4 transcription factor activator gene; thereby forming a GVG inducible system or a VGE inducible system.

In another embodiment, the lexA-dependent effector is at least one LexA operon. In one aspect, the method further comprises an additional expression cassette comprising a nucleotide sequence encoding a LexA:VP16:ER activator; thereby forming an XVE inducible system.

In one embodiment, the DNA modification enzyme is selected from the group consisting of a meganuclease (MN), a zinc-finger nuclease (ZFN), a transcription-activator like effector nuclease (TALEN), a chimeric FEN1-FokI, a Mega-TALs, and a CRISPR nuclease. In one aspect, the CRISPR nuclease is a Cas nuclease, a Cas9 nuclease, a Cpf1 nuclease, a dCas9-FokI, a dCpf1-FokI, a chimeric Cas9-cytidine deaminase, a chimeric Cas9-adenine deaminase, a nickase Cas9 (nCas9), a chimeric dCas9 non-FokI nuclease, and a dCpf1 non-FokI nuclease, a Cas12a fused to a deaminase domain, a Cas12i nuclease, a Cas12j nuclease, a CasX nuclease, a CasY nuclease, a Cas13 nuclease, a Cas14 nuclease.

In another embodiment, the translocation factor is a glucocorticoid receptor. In one aspect, the glucocorticoid receptor comprises SEQ ID NO:6. In another aspect, the glucocorticoid receptor is operably linked to a CRISPR nuclease. In another embodiment, the glucocorticoid receptor-linked CRISPR nuclease is a modified Cas12a nuclease modified to comprise a glucocorticoid receptor binding domain (“GR-Cas12a”). In one aspect, the GR-Cas12a comprises SEQ ID NO: 7. In another embodiment, the method further comprises, upon application of dexamethasone, the GR-Cas12a translocates from the cytoplasm to the nucleus of the plant cell or plant tissue.

In another embodiment of the method, the unique edit is an indel mutation, a nucleotide substitution, an allele replacement, a chromosomal translocation, or an insertion of donor nucleic acid.

In another embodiment of the method, the plant cell or plant tissue is dicotyledonous. In one aspect, the dicotyledonous plant cell or plant tissue is selected from the group consisting of Arabidopsis, sunflower, soybean, tomato, Brassica species, Populus (poplar), Eucalyptus, tobacco, Cannabis, potato, cotton, maize, rice, wheat, barley, sugarcane, Glycine tomentella, and other wild Glycine species.

In another embodiment of the method, the plant cell or plant tissue is monocotyledonous. In one aspect, the monocotyledonous plant cell or plant tissue is selected from the group consisting of maize, wheat, rice, teosinte, sorghum, barley. In another aspect, the monocotyledonous plant cell or plant tissue is maize.

In one embodiment, plant cell or plant tissue is maize and wherein the desired developmental stage is selected from the group consisting of VE, V1, V2, V(n), VT, R1, R2, R3, R4, R5, and R6 stage; where (n) is an integer representing the number of leaf collars present.

In another embodiment, plant cell or plant tissue is soybean and wherein the desired developmental stage is selected from the group consisting of VE, VC, V1, V2, V(n), R1, R2, R3, R4, R5, R6, R7, and R8 stage; where (n) is an integer representing the number of trifoliolates present.

In another embodiment, the method further comprises (d.) growing the progeny collectively comprising a plurality of unique edits into seedlings, plantlets, immature plants, mature plants, or senescent plants; (e.) measuring at least one phenotype in the seedlings, plantlets, immature plants, mature plants, or senescent plants of step a.; and (f.) optionally selecting a seedling, plantlet, immature plant, mature plant, or senescent plant based on the measuring of the at least one phenotype.

In yet another embodiment, the method further comprises (d.) growing the progeny collectively comprising a plurality of unique edits into seedlings, plantlets, immature plants, mature plants, or senescent plants; (e.) genotyping the seedlings, plantlets, immature plants, mature plants, or senescent plants of step a.; and (f.) optionally selecting a seedling, plantlet, immature plant, mature plant, or senescent plant based on the genotype of step b.

Another embodiment of the invention is an edited plant produced by the methods recited above.

Another embodiment of the invention is an inducible gene editing system, comprising an expression cassette comprising (a.) a nucleic acid encoding a DNA modification enzyme; (b.) an optional nucleic acid encoding at least one guide RNA; and (c.) an inducible factor operably linked to the nucleic acid encoding a DNA modification enzyme. In one embodiment, the system further comprises a cell harboring the expression cassette. In one aspect, the cell is a eukaryotic cell. In another aspect, the eukaryotic cell is a plant cell.

### EXAMPLES

### Example 1. Alcohol-Induced Mosaicism

In this example, mosaicism can be induced by application of ethanol to a plant comprising the AlcR/AlcA inducible system operably linked to a GE system at a desired developmental stage of plant life, e.g., development of the floral primordia. In the AlcR/AlcA system, the AlcR transcription factor and the AlcA promoter were isolated from Aspergillum nidulans. When a plant comprising this system is exposed to ethanol, the plant metabolizes ethanol into acetaldehyde, which in conjunction with AlcR activates the AlcA promoter, thus driving expression of the downstream gene.

Materials used: (1) Two chambers at 28° C. during induction and for two weeks. (2) Arabidopsis plants were transformed with vector 25881, comprising an ethanol-inducible gene-editing system for expression in Arabidopsis with kanamycin selection marker that includes three cassettes. In the first expression cassette, a dicot-optimized alcohol receptor gene (AlcR) from Aspergillus nidulans is driven by the constitutive promoter prAtEF1aA1. In the second expression cassette, prAlcA, a chimeric promoter consisting of a fusion of AlcA promoter and a 35S minimal promoter (described in Caddick et al, 1998. Nature Biotechnology) drives expression of Cas12a. In the presence of ethanol, AlcR binds to the AlcA promoter and activates transcription. The third expression cassette comprises the gRNA targeting the second exon of Glabrous1 (GL1) gene.

Treatments: (1) Overnight drench with a 2% ethanol water solution. (2) The control plants were grown under the same conditions but did not receive ethanol, they were drenched with water only.

Sampling for edits: 8-16 siliques from various parts of the plant harvested, seeds germinated, and plants sampled for sequencing, all seeds from one silique go into the same pot. Vernalized for two days at 4 C after seeds are planted in soil.

After bolting it takes about a month for the first siliques to be ready for harvest.

### A. Drench Treatment No. 1

Plants were drenched with 2% ethanol. Controls were kept in a separate chamber without ethanol.

### B. qRT PCR Results

We initially tested the levels of Cas12a transcript the day after overnight drench with 2% ethanol (17 hours) and after 6 days (144 hours), this experiment was named ‘1_6_days’. We found that Cas12a was induced 17 hours after the beginning of the drench with 2% ethanol but it was back to water-control levels after 144 hours. In order to better estimate the expression profile over time of induced Cas12a transcript we performed another experiment, named ‘Timecourse’, with a second batch of 25881 Arabidopsis plants, sampled at 17, 46, 70, and 94 hours after drenching. In the second experiment (‘Timecourse’), the trays containing the control plants were placed next to those drenched with 2% ethanol. After 17 hours the control plants showed activation of Cas12a transcript, ostensibly from ethanol vapor coming from the 2% alc tray. For this reason, water control data points for day 1 (17 hours) from the ‘Timecourse’ experiment were excluded from the analysis.

We quantified the levels of Cas12a transcript using a TaqMan qRT-PCR (Table 1.). Based on the results from these experiments we chose to drench with 2% ethanol every 4 days to maintain Cas12a induced.

TABLE 1
Quantified levels of Cas12a expression

| | | Time | | |
|---|---|---|---|---|
| PlantID | Treatment | (hours) | Cas12a* | Experiment |
| Plant_01 | 2% alcohol | 17 | 8417 | Timecourse |
| Plant_02 | 2% alcohol | 17 | 7807 | Timecourse |
| Plant_03 | 2% alcohol | 17 | 212 | Timecourse |
| Plant_04 | 2% alcohol | 17 | 3170 | Timecourse |
| Plant_05 | 2% alcohol | 17 | 3129 | Timecourse |
| Plant_06 | 2% alcohol | 17 | 5033 | Timecourse |
| Plant_07 | 2% alcohol | 17 | 6875 | Timecourse |
| Plant_08 | 2% alcohol | 17 | 751 | Timecourse |
| Plant_09 | 2% alcohol | 17 | 2207 | Timecourse |
| Plant_10 | 2% alcohol | 17 | 5622 | Timecourse |
| Plant_11 | 2% alcohol | 17 | 539 | Timecourse |
| Plant_12 | 2% alcohol | 17 | 5374 | Timecourse |
| Plant_13 | 2% alcohol | 17 | 130 | Timecourse |
| Plant_14 | 2% alcohol | 17 | 3935 | Timecourse |
| Plant_15 | 2% alcohol | 17 | 27544 | Timecourse |
| Plant_16 | 2% alcohol | 17 | NA | Timecourse |
| Plant_17 | 2% alcohol | 17 | 14149 | Timecourse |
| Plant_18 | 2% alcohol | 17 | 3847 | Timecourse |
| Plant_19 | 2% alcohol | 17 | 561 | Timecourse |
| Plant_20 | 2% alcohol | 17 | 47 | Timecourse |
| Plant_21 | 2% alcohol | 17 | 1442 | Timecourse |
| Plant_22 | 2% alcohol | 17 | 2926 | Timecourse |
| Plant_23 | 2% alcohol | 17 | 7666 | Timecourse |
| Plant_24 | 2% alcohol | 17 | 5444 | Timecourse |
| UR252260506 | 2% alcohol | 17 | 47797 | 1_6_days |
| UR252260507 | 2% alcohol | 17 | 318 | 1_6_days |
| UR252260508 | 2% alcohol | 17 | 79385 | 1_6_days |
| UR252260509 | 2% alcohol | 17 | 45084 | 1_6_days |
| UR252260510 | 2% alcohol | 17 | 94136 | 1_6_days |
| UR252260511 | 2% alcohol | 17 | 123797 | 1_6_days |
| UR252260512 | 2% alcohol | 17 | 42454 | 1_6_days |
| UR252260513 | 2% alcohol | 17 | 85180 | 1_6_days |
| UR252260514 | 2% alcohol | 17 | 48364 | 1_6_days |
| UR252260515 | 2% alcohol | 17 | 19354 | 1_6_days |
| UR252260517 | 2% alcohol | 17 | 58140 | 1_6_days |
| UR252260518 | 2% alcohol | 17 | 54815 | 1_6_days |
| UR252260519 | 2% alcohol | 17 | 22214 | 1_6_days |
| UR252260522 | 2% alcohol | 17 | 115912 | 1_6_days |
| UR252260523 | 2% alcohol | 17 | 41347 | 1_6_days |
| UR252260524 | 2% alcohol | 17 | 2120 | 1_6_days |
| UR252260525 | 2% alcohol | 17 | 33943 | 1_6_days |
| UR252260527 | 2% alcohol | 17 | 4033 | 1_6_days |
| UR252260529 | 2% alcohol | 17 | 62058 | 1_6_days |
| UR252260530 | 2% alcohol | 17 | 21924 | 1_6_days |
| UR252260531 | 2% alcohol | 17 | 125600 | 1_6_days |
| UR252260532 | 2% alcohol | 17 | 48957 | 1_6_days |
| UR252260537 | 2% alcohol | 17 | 92009 | 1_6_days |
| UR252260538 | 2% alcohol | 17 | 11939 | 1_6_days |
| UR252260540 | 2% alcohol | 17 | 60385 | 1_6_days |
| UR252260541 | 2% alcohol | 17 | 16 | 1_6_days |
| UR252260542 | 2% alcohol | 17 | 207 | 1_6_days |
| UR252260548 | 2% alcohol | 17 | 73393 | 1_6_days |
| UR252260549 | 2% alcohol | 17 | 31067 | 1_6_days |
| UR252260555 | 2% alcohol | 17 | 36 | 1_6_days |
| UR252260556 | 2% alcohol | 17 | 184518 | 1_6_days |
| UR252260562 | 2% alcohol | 17 | 34468 | 1_6_days |
| UR252260563 | 2% alcohol | 17 | 147126 | 1_6_days |
| UR252260564 | 2% alcohol | 17 | 129700 | 1_6_days |
| UR252260565 | 2% alcohol | 17 | 34442 | 1_6_days |
| UR252260567 | 2% alcohol | 17 | 36581 | 1_6_days |
| UR252260568 | 2% alcohol | 17 | 74545 | 1_6_days |
| UR252260569 | 2% alcohol | 17 | 14910 | 1_6_days |
| UR252260570 | 2% alcohol | 17 | 35470 | 1_6_days |
| UR252260571 | 2% alcohol | 17 | 2565 | 1_6_days |
| UR252260572 | 2% alcohol | 17 | 54570 | 1_6_days |
| UR252260573 | 2% alcohol | 17 | 1796 | 1_6_days |
| UR252260574 | 2% alcohol | 17 | 6453 | 1_6_days |
| UR252260575 | 2% alcohol | 17 | 17026 | 1_6_days |
| UR252260576 | 2% alcohol | 17 | 36941 | 1_6_days |
| UR252260577 | 2% alcohol | 17 | 84090 | 1_6_days |
| UR252260578 | 2% alcohol | 17 | 55295 | 1_6_days |
| UR252260579 | 2% alcohol | 17 | 65526 | 1_6_days |
| UR252260580 | 2% alcohol | 17 | 30212 | 1_6_days |
| UR252260581 | 2% alcohol | 17 | 364 | 1_6_days |
| UR252260582 | 2% alcohol | 17 | 18798 | 1_6_days |
| UR252260584 | 2% alcohol | 17 | 194394 | 1_6_days |
| UR252260586 | 2% alcohol | 17 | 21544 | 1_6_days |
| UR252260587 | 2% alcohol | 17 | 41755 | 1_6_days |
| UR252260588 | 2% alcohol | 17 | 20035 | 1_6_days |
| UR252260589 | 2% alcohol | 17 | 39733 | 1_6_days |
| UR252260591 | 2% alcohol | 17 | 20375 | 1_6_days |
| UR252260594 | 2% alcohol | 17 | 105874 | 1_6_days |
| UR252260595 | 2% alcohol | 17 | 249984 | 1_6_days |
| UR252260596 | 2% alcohol | 17 | 27649 | 1_6_days |
| UR252260598 | 2% alcohol | 17 | 53140 | 1_6_days |
| UR252260599 | 2% alcohol | 17 | 48317 | 1_6_days |
| UR252260600 | 2% alcohol | 17 | 44923 | 1_6_days |
| UR252260601 | 2% alcohol | 17 | 98382 | 1_6_days |
| UR252260602 | 2% alcohol | 17 | 23250 | 1_6_days |
| UR252260603 | 2% alcohol | 17 | 235136 | 1_6_days |
| UR252260604 | 2% alcohol | 17 | 66600 | 1_6_days |
| UR252260505 | Water | 17 | 326 | 1_6_days |
| UR252260516 | Water | 17 | 33 | 1_6_days |
| UR252260521 | Water | 17 | 249 | 1_6_days |
| UR252260528 | Water | 17 | 0 | 1_6_days |
| UR252260535 | Water | 17 | 640 | 1_6_days |
| UR252260536 | Water | 17 | 48 | 1_6_days |
| UR252260539 | Water | 17 | 45 | 1_6_days |
| UR252260544 | Water | 17 | 722 | 1_6_days |
| UR252260545 | Water | 17 | 130 | 1_6_days |
| UR252260546 | Water | 17 | 1355 | 1_6_days |
| UR252260547 | Water | 17 | 0 | 1_6_days |
| UR252260550 | Water | 17 | 6 | 1_6_days |
| UR252260551 | Water | 17 | 644 | 1_6_days |
| UR252260552 | Water | 17 | 0 | 1_6_days |
| UR252260553 | Water | 17 | 0 | 1_6_days |
| UR252260554 | Water | 17 | 13 | 1_6_days |
| UR252260557 | Water | 17 | 1 | 1_6_days |
| UR252260558 | Water | 17 | 120 | 1_6_days |
| UR252260559 | Water | 17 | 423 | 1_6_days |
| UR252260560 | Water | 17 | 179 | 1_6_days |
| UR252260561 | Water | 17 | 0 | 1_6_days |
| Plant_01 | 2% alcohol | 46 | 24212 | Timecourse |
| Plant_02 | 2% alcohol | 46 | 18800 | Timecourse |
| Plant_03 | 2% alcohol | 46 | 1186 | Timecourse |
| Plant_04 | 2% alcohol | 46 | 9085 | Timecourse |
| Plant_05 | 2% alcohol | 46 | 16446 | Timecourse |
| Plant_06 | 2% alcohol | 46 | 9503 | Timecourse |
| Plant_07 | 2% alcohol | 46 | 5569 | Timecourse |
| Plant_08 | 2% alcohol | 46 | 4959 | Timecourse |
| Plant_09 | 2% alcohol | 46 | 15609 | Timecourse |
| Plant_10 | 2% alcohol | 46 | 9455 | Timecourse |
| Plant_11 | 2% alcohol | 46 | 5753 | Timecourse |
| Plant_12 | 2% alcohol | 46 | 33869 | Timecourse |
| Plant_13 | 2% alcohol | 46 | 561 | Timecourse |
| Plant_14 | 2% alcohol | 46 | 15316 | Timecourse |
| Plant_15 | 2% alcohol | 46 | 15944 | Timecourse |
| Plant_16 | 2% alcohol | 46 | 7943 | Timecourse |
| Plant_17 | 2% alcohol | 46 | 23241 | Timecourse |
| Plant_18 | 2% alcohol | 46 | 23530 | Timecourse |
| Plant_19 | 2% alcohol | 46 | 760 | Timecourse |
| Plant_20 | 2% alcohol | 46 | 1487 | Timecourse |
| Plant_21 | 2% alcohol | 46 | 13040 | Timecourse |
| Plant_22 | 2% alcohol | 46 | 12840 | Timecourse |
| Plant_23 | 2% alcohol | 46 | 39288 | Timecourse |
| Plant_24 | 2% alcohol | 46 | 5958 | Timecourse |
| Plant_33 | Water | 46 | 418 | Timecourse |
| Plant_34 | Water | 46 | 13 | Timecourse |
| Plant_35 | Water | 46 | 0 | Timecourse |
| Plant_36 | Water | 46 | 45 | Timecourse |
| Plant_37 | Water | 46 | 153 | Timecourse |
| Plant_38 | Water | 46 | 77 | Timecourse |
| Plant_39 | Water | 46 | 20 | Timecourse |
| Plant_40 | Water | 46 | 0 | Timecourse |
| Plant_01 | 2% alcohol | 70 | 22461 | Timecourse |
| Plant_01 | 2% alcohol | 70 | 19972 | Timecourse |
| Plant_02 | 2% alcohol | 70 | 74627 | Timecourse |
| Plant_02 | 2% alcohol | 70 | 30704 | Timecourse |
| Plant_03 | 2% alcohol | 70 | 1603 | Timecourse |
| Plant_03 | 2% alcohol | 70 | 418 | Timecourse |
| Plant_04 | 2% alcohol | 70 | 9514 | Timecourse |
| Plant_04 | 2% alcohol | 70 | 4094 | Timecourse |
| Plant_05 | 2% alcohol | 70 | 11731 | Timecourse |
| Plant_05 | 2% alcohol | 70 | 7035 | Timecourse |
| Plant_06 | 2% alcohol | 70 | 19944 | Timecourse |
| Plant_06 | 2% alcohol | 70 | 11952 | Timecourse |
| Plant_07 | 2% alcohol | 70 | 6660 | Timecourse |
| Plant_07 | 2% alcohol | 70 | 13822 | Timecourse |
| Plant_08 | 2% alcohol | 70 | 1258 | Timecourse |
| Plant_08 | 2% alcohol | 70 | 1240 | Timecourse |
| Plant_09 | 2% alcohol | 70 | 20985 | Timecourse |
| Plant_09 | 2% alcohol | 70 | 12621 | Timecourse |
| Plant_10 | 2% alcohol | 70 | 19152 | Timecourse |
| Plant_10 | 2% alcohol | 70 | 5588 | Timecourse |
| Plant_11 | 2% alcohol | 70 | 3628 | Timecourse |
| Plant_11 | 2% alcohol | 70 | 3114 | Timecourse |
| Plant_12 | 2% alcohol | 70 | 96736 | Timecourse |
| Plant_12 | 2% alcohol | 70 | 11749 | Timecourse |
| Plant_13 | 2% alcohol | 70 | 1853 | Timecourse |
| Plant_13 | 2% alcohol | 70 | 1214 | Timecourse |
| Plant_14 | 2% alcohol | 70 | 15044 | Timecourse |
| Plant_14 | 2% alcohol | 70 | 11601 | Timecourse |
| Plant_15 | 2% alcohol | 70 | 30317 | Timecourse |
| Plant_15 | 2% alcohol | 70 | 18518 | Timecourse |
| Plant_16 | 2% alcohol | 70 | 15832 | Timecourse |
| Plant_16 | 2% alcohol | 70 | 12206 | Timecourse |
| Plant_17 | 2% alcohol | 70 | 42737 | Timecourse |
| Plant_18 | 2% alcohol | 70 | 27310 | Timecourse |
| Plant_19 | 2% alcohol | 70 | 8763 | Timecourse |
| Plant_20 | 2% alcohol | 70 | 606 | Timecourse |
| Plant_21 | 2% alcohol | 70 | 16814 | Timecourse |
| Plant_22 | 2% alcohol | 70 | 13588 | Timecourse |
| Plant_23 | 2% alcohol | 70 | 49320 | Timecourse |
| Plant_24 | 2% alcohol | 70 | 16111 | Timecourse |
| Plant_33 | Water | 70 | 879 | Timecourse |
| Plant_34 | Water | 70 | 0 | Timecourse |
| Plant_35 | Water | 70 | 12 | Timecourse |
| Plant_36 | Water | 70 | 45 | Timecourse |
| Plant_37 | Water | 70 | 39 | Timecourse |
| Plant_38 | Water | 70 | 21 | Timecourse |
| Plant_39 | Water | 70 | 1 | Timecourse |
| Plant_40 | Water | 70 | 2 | Timecourse |
| Plant_01 | 2% alcohol | 94 | 9046 | Timecourse |
| Plant_02 | 2% alcohol | 94 | 5556 | Timecourse |
| Plant_03 | 2% alcohol | 94 | 11 | Timecourse |
| Plant_04 | 2% alcohol | 94 | 1760 | Timecourse |
| Plant_05 | 2% alcohol | 94 | 3459 | Timecourse |
| Plant_06 | 2% alcohol | 94 | 8870 | Timecourse |
| Plant_07 | 2% alcohol | 94 | 4105 | Timecourse |
| Plant_08 | 2% alcohol | 94 | 116 | Timecourse |
| Plant_09 | 2% alcohol | 94 | 35997 | Timecourse |
| Plant_10 | 2% alcohol | 94 | 12986 | Timecourse |
| Plant_11 | 2% alcohol | 94 | 5062 | Timecourse |
| Plant_12 | 2% alcohol | 94 | 27845 | Timecourse |
| Plant_13 | 2% alcohol | 94 | 200 | Timecourse |
| Plant_14 | 2% alcohol | 94 | 6216 | Timecourse |
| Plant_15 | 2% alcohol | 94 | 34693 | Timecourse |
| Plant_16 | 2% alcohol | 94 | 2945 | Timecourse |
| Plant_17 | 2% alcohol | 94 | 92791 | Timecourse |
| Plant_18 | 2% alcohol | 94 | 27471 | Timecourse |
| Plant_19 | 2% alcohol | 94 | 1789 | Timecourse |
| Plant_20 | 2% alcohol | 94 | 481 | Timecourse |
| Plant_21 | 2% alcohol | 94 | 13801 | Timecourse |
| Plant_22 | 2% alcohol | 94 | 34079 | Timecourse |
| Plant_23 | 2% alcohol | 94 | 9986 | Timecourse |
| Plant_24 | 2% alcohol | 94 | 2474 | Timecourse |
| Plant_33 | Water | 94 | 1705 | Timecourse |
| Plant_34 | Water | 94 | 6 | Timecourse |
| Plant_35 | Water | 94 | 20 | Timecourse |
| Plant_36 | Water | 94 | 29 | Timecourse |
| Plant_37 | Water | 94 | 210 | Timecourse |
| Plant_38 | Water | 94 | 42 | Timecourse |
| Plant_39 | Water | 94 | 9 | Timecourse |
| Plant_40 | Water | 94 | 4 | Timecourse |
| UR252260506 | 2% alcohol | 144 | 147 | 1_6_days |
| UR252260509 | 2% alcohol | 144 | 426 | 1_6_days |
| UR252260510 | 2% alcohol | 144 | 1027 | 1_6_days |
| UR252260513 | 2% alcohol | 144 | 174 | 1_6_days |
| UR252260514 | 2% alcohol | 144 | 10 | 1_6_days |
| UR252260515 | 2% alcohol | 144 | 14 | 1_6_days |
| UR252260518 | 2% alcohol | 144 | 3275 | 1_6_days |
| UR252260523 | 2% alcohol | 144 | 34 | 1_6_days |
| UR252260524 | 2% alcohol | 144 | 0 | 1_6_days |
| UR252260527 | 2% alcohol | 144 | 0 | 1_6_days |
| UR252260529 | 2% alcohol | 144 | 22 | 1_6_days |
| UR252260531 | 2% alcohol | 144 | 762 | 1_6_days |
| UR252260533 | 2% alcohol | 144 | 6 | 1_6_days |
| UR252260548 | 2% alcohol | 144 | 44 | 1_6_days |
| UR252260549 | 2% alcohol | 144 | 8 | 1_6_days |
| UR252260564 | 2% alcohol | 144 | 1256 | 1_6_days |
| UR252260565 | 2% alcohol | 144 | 0 | 1_6_days |
| UR252260569 | 2% alcohol | 144 | 21 | 1_6_days |
| UR252260572 | 2% alcohol | 144 | 1028 | 1_6_days |
| UR252260573 | 2% alcohol | 144 | 0 | 1_6_days |
| UR252260575 | 2% alcohol | 144 | 0 | 1_6_days |
| UR252260576 | 2% alcohol | 144 | 35 | 1_6_days |
| UR252260577 | 2% alcohol | 144 | 299 | 1_6_days |
| UR252260579 | 2% alcohol | 144 | 51 | 1_6_days |
| UR252260580 | 2% alcohol | 144 | 846 | 1_6_days |
| UR252260584 | 2% alcohol | 144 | 711 | 1_6_days |
| UR252260597 | 2% alcohol | 144 | 121 | 1_6_days |
| UR252260598 | 2% alcohol | 144 | 2622 | 1_6_days |
| UR252260601 | 2% alcohol | 144 | 687 | 1_6_days |
| UR252260528 | Water | 144 | 1 | 1_6_days |
| UR252260536 | Water | 144 | 122 | 1_6_days |
| UR252260546 | Water | 144 | 1754 | 1_6_days |
| UR252260547 | Water | 144 | 1 | 1_6_days |
| UR252260551 | Water | 144 | 133 | 1_6_days |
| UR252260554 | Water | 144 | 22 | 1_6_days |
| UR252260558 | Water | 144 | 59 | 1_6_days |
| UR252260559 | Water | 144 | 22 | 1_6_days |

*×1000 relative to endogenous control

We also performed an experiment to compare expression of Cas12a in flowers and leaves. This experiment was done using T2 plants from a subset of T1 plants. The data shows that alcohol induces Cas12a expression systemically (in leaves and flowers).

TABLE 2
Comparing Cas12a expression in leaf and flower tissues.

| T1.PlantID | T2.PlantID | Tissue | Cas12a.* |
|---|---|---|---|
| UR259223922 | 2 | Leaf | 8829 |
| UR259223922 | 2 | Flower | 8288 |
| UR259223922 | 5 | Leaf | 248803 |
| UR259223922 | 5 | Flower | 67593 |
| UR259223922 | 6 | Leaf | 91370 |
| UR259223922 | 6 | Flower | 59885 |
| UR259223922 | 7 | Leaf | 92605 |
| UR259223922 | 7 | Flower | 83025 |
| UR259223922 | 17 | Leaf | 89259 |
| UR259223922 | 17 | Flower | 58486 |
| UR259223922 | 21 | Leaf | 211321 |
| UR259223922 | 21 | Flower | 106951 |
| UR259223927 | 3 | Leaf | 73102 |
| UR259223927 | 3 | Flower | 52055 |
| UR259223927 | 9 | Leaf | 71500 |
| UR259223927 | 9 | Flower | 54198 |
| UR259223927 | 12 | Leaf | 71460 |
| UR259223927 | 12 | Flower | 20319 |
| UR259223927 | 17 | Leaf | 88207 |
| UR259223927 | 17 | Flower | 25141 |
| UR259223927 | 19 | Leaf | 101975 |
| UR259223927 | 19 | Flower | 41739 |
| UR259223928 | 1 | Flower | 12110 |
| UR259223928 | 1 | Leaf | 26841 |
| UR259223928 | 3 | Flower | 39204 |
| UR259223928 | 3 | Leaf | 42432 |
| UR259223928 | 6 | Flower | 76038 |
| UR259223928 | 6 | Leaf | 51415 |
| UR259223928 | 7 | Flower | 27906 |
| UR259223928 | 7 | Leaf | 59705 |
| UR259223928 | 8 | Flower | 49917 |
| UR259223928 | 8 | Leaf | ND |
| UR259223928 | 10 | Flower | 39721 |
| UR259223928 | 10 | Leaf | 45642 |
| UR259223928 | 12 | Flower | 29341 |
| UR259223928 | 12 | Leaf | 18426 |
| UR259223928 | 13 | Flower | 36175 |
| UR259223928 | 13 | Leaf | 29629 |
| UR259223928 | 14 | Flower | 39525 |
| UR259223928 | 14 | Leaf | 71367 |
| UR259223928 | 16 | Flower | 28584. |
| UR259223928 | 16 | Leaf | 73449 |
| UR259223928 | 17 | Flower | 31574 |
| UR259223928 | 17 | Leaf | 39270 |
| UR259223928 | 18 | Flower | 33850 |
| UR259223928 | 18 | Leaf | 35495 |
| UR259223928 | 19 | Flower | 41077 |
| UR259223928 | 19 | Leaf | 47672 |
| UR259223928 | 20 | Flower | 50834 |
| UR259223928 | 20 | Leaf | 40061 |
| UR259223928 | 24 | Flower | 13577 |
| UR259223928 | 24 | Leaf | 60033 |
| UR259223928 | 26 | Flower | 36352 |
| UR259223928 | 26 | Leaf | 45621 |
| UR259223928 | 27 | Flower | 44569 |
| UR259223928 | 27 | Leaf | 53181 |
| UR259223928 | 28 | Flower | 43725 |
| UR259223928 | 28 | Leaf | 28241 |
| UR259223928 | 29 | Flower | 29512 |
| UR259223928 | 29 | Leaf | 35509 |
| UR259223928 | 30 | Flower | 49429 |
| UR259223928 | 30 | Leaf | 38253 |
| UR259223928 | 31 | Flower | 47448 |
| UR259223928 | 31 | Leaf | 27681 |
| UR259223948 | 1 | Leaf | 123179 |
| UR259223948 | 1 | Flower | 99750 |
| UR259223948 | 10 | Leaf | 144957 |
| UR259223948 | 10 | Flower | 42583 |
| UR259223948 | 12 | Leaf | 166935 |
| UR259223948 | 12 | Flower | 47038 |
| UR259223948 | 15 | Leaf | 89807 |
| UR259223948 | 15 | Flower | 90142 |
| UR259223952 | 3 | Leaf | 6359 |
| UR259223952 | 3 | Flower | 4461 |
| UR259223952 | 4 | Leaf | 8438 |
| UR259223952 | 4 | Flower | 5507 |
| UR259223952 | 6 | Leaf | 3277 |
| UR259223952 | 6 | Flower | 9455 |
| UR259223952 | 10 | Leaf | 7837 |
| UR259223952 | 10 | Flower | 7840 |
| UR259223952 | 11 | Leaf | 57060 |
| UR259223952 | 11 | Flower | 21370 |
| UR259223952 | 12 | Leaf | 5735 |
| UR259223952 | 12 | Flower | 9939 |
| UR259223952 | 13 | Leaf | 27784 |
| UR259223952 | 13 | Flower | 22455 |
| UR259223952 | 20 | Leaf | 5716 |
| UR259223952 | 20 | Flower | 5454 |
| UR259223952 | 21 | Leaf | 2195 |
| UR259223952 | 21 | Flower | 4851 |
| UR259223952 | 22 | Leaf | 6051 |
| UR259223952 | 22 | Flower | 32977 |
| UR259223952 | 24 | Leaf | 55377 |
| UR259223952 | 24 | Flower | 24695 |
| UR259223960 | 1 | Leaf | 0 |
| UR259223960 | 1 | Flower | 0 |
| UR259223960 | 8 | Leaf | 67575 |
| UR259223960 | 8 | Flower | 30119 |
| UR259223960 | 9 | Leaf | 171502 |
| UR259223960 | 9 | Flower | 95798 |
| UR259223960 | 10 | Leaf | 160533 |
| UR259223960 | 10 | Flower | 92743 |
| UR259223960 | 11 | Leaf | 107654 |
| UR259223960 | 11 | Flower | 36948 |
| UR259223960 | 14 | Leaf | 42767 |
| UR259223960 | 14 | Flower | 25505 |
| UR259223960 | 15 | Leaf | 68035 |
| UR259223960 | 15 | Flower | 93269 |
| UR259223960 | 16 | Leaf | 148512 |
| UR259223960 | 16 | Flower | 102110 |
| UR259223960 | 18 | Leaf | 27547 |
| UR259223960 | 18 | Flower | 68117 |
| UR259223960 | 19 | Leaf | 44873 |
| UR259223960 | 19 | Flower | 34274 |
| UR259223960 | 20 | Leaf | 57752 |
| UR259223960 | 20 | Flower | 42522 |
| UR259223960 | 21 | Leaf | 77560 |
| UR259223960 | 21 | Flower | 37693 |
| UR259223960 | 22 | Leaf | 22386 |
| UR259223960 | 22 | Flower | 32155 |
| UR259223960 | 30 | Leaf | 0 |
| UR259223960 | 30 | Flower | 0 |
| UR259223960 | 34 | Leaf | 24060 |
| UR259223960 | 34 | Flower | 25709 |
| UR259223960 | 37 | Leaf | ND |
| UR259223960 | 37 | Flower | 22911 |
| UR259223961 | 1 | Flower | 28380 |
| UR259223961 | 1 | Leaf | 8716 |
| UR259223961 | 2 | Flower | 51762 |
| UR259223961 | 2 | Leaf | 10226 |
| UR259223961 | 3 | Flower | 53351 |
| UR259223961 | 3 | Leaf | 28644. |
| UR259223961 | 4 | Flower | 29305 |
| UR259223961 | 4 | Leaf | 1938 |
| UR259223961 | 5 | Flower | 24358 |
| UR259223961 | 5 | Leaf | 7512 |
| UR259223961 | 6 | Flower | 53455 |
| UR259223961 | 6 | Leaf | 25565 |
| UR259223961 | 8 | Flower | 22951 |
| UR259223961 | 8 | Leaf | 14735 |
| UR259223961 | 9 | Flower | 50627 |
| UR259223961 | 9 | Leaf | 12143 |
| UR259223961 | 10 | Flower | 65972 |
| UR259223961 | 10 | Leaf | 7092 |
| UR259223961 | 12 | Flower | 25137 |
| UR259223961 | 12 | Leaf | 9171 |
| UR259223961 | 13 | Flower | 49385 |
| UR259223961 | 13 | Leaf | 8448 |
| UR259223961 | 14 | Flower | 8178 |
| UR259223961 | 14 | Leaf | 3279 |
| UR259223961 | 17 | Flower | 3630 |
| UR259223961 | 17 | Leaf | 9808 |
| UR259223961 | 19 | Flower | 54093 |
| UR259223961 | 19 | Leaf | 28329 |
| UR259223961 | 20 | Flower | 2780 |
| UR259223961 | 20 | Leaf | 1641 |
| UR259223961 | 21 | Flower | 28371 |
| UR259223961 | 21 | Leaf | 5560 |
| UR259223961 | 22 | Flower | 35384 |
| UR259223961 | 22 | Leaf | 9034 |
| UR259223961 | 23 | Flower | 56894 |
| UR259223961 | 23 | Leaf | 17570 |
| UR259223961 | 24 | Flower | 5232 |
| UR259223961 | 24 | Leaf | 4531 |
| UR259223961 | 25 | Flower | 20925 |
| UR259223961 | 25 | Leaf | 5250 |
| UR259223961 | 33 | Flower | 55792 |
| UR259223961 | 33 | Leaf | 31619 |

*×1000 relative to endogenous control

### C. Expression Timeline

### Cas12a Alcohol Induction and gl1 Mutagenesis

To optimize the rate of gl1 mutagenesis we divided the T1 events into four batches to be induced at different times after planting. Plants in the First batch were induced 23 days after transplanting to soil, while they were all in the vegetative stage. Plants in the Second batch were induced 34 days after transplanting, at which time all but two were in vegetative stage. Plants in the Third batch were induced 44 days after transplanting and were all flowering at this time. Plants in the Fourth batch were induced 47 days after transplanting. All plants were drenched every four days after their initial induction to maintain Cas12a activated. Leaf samples were taken before induction and at one day after induction for all plants. In addition, some plants were sampled to measure Cas12a at later timepoints.

Table 3, showing the expression levels of Cas12a, relative to an endogenous control, at different times before and after the first induction with alcohol.

| | Days after | | | |
|---|---|---|---|---|
| PlantID | transplanting | | Cas12a* | Treatment |
| UR259223920 | uninduced | | 0 | First |
| | 1 | day | 1654 | |
| | 9 | days | 41177 | |
| UR259223944 | uninduced | | 588 | |
| | 1 | day | 8194 | |
| UR259223958 | uninduced | | 0 | |
| | 1 | day | 130 | |
| UR259223959 | uninduced | | 0 | |
| | 1 | day | 18860 | |
| | 9 | days | 31854 | |
| UR259223961 | uninduced | | 0 | |
| | 1 | day | 10601 | |
| | 9 | days | 21862 | |
| UR259223962 | uninduced | | 0 | |
| | 1 | day | 1858 | |
| | 9 | days | 43458 | |
| UR259223963 | uninduced | | 0 | |
| | 1 | day | 5340 | |
| | 9 | days | 35641 | |
| UR259223964 | uninduced | | 191 | |
| | 1 | day | 10354 | |
| | 9 | days | 57548 | |
| UR259223903 | uninduced | | 402 | Second |
| | 1 | day | 27830 | |
| | 13 | days | 22921 | |
| | 20 | days | 7362 | |
| UR259223905 | uninduced | | 0 | |
| | 1 | day | 6028 | |
| | 13 | days | 24704 | |
| | 20 | days | 965 | |
| UR259223906 | uninduced | | 235 | |
| | 1 | day | 14101 | |
| | 13 | days | 31215 | |
| | 20 | days | 21616 | |
| UR259223907 | uninduced | | 55 | |
| | 1 | day | 7876 | |
| | 13 | days | 30349 | |
| | 20 | days | 7512 | |
| UR259223909 | uninduced | | 0 | |
| | 1 | day | 4631 | |
| | 13 | days | 30102 | |
| | 20 | days | 7562 | |
| UR259223910 | uninduced | | 9 | |
| | 1 | day | 22474 | |
| | 13 | days | 37733 | |
| | 20 | days | 1351 | |
| UR259223917 | uninduced | | 24 | |
| | 1 | day | 39386 | |
| | 13 | days | 23346 | |
| | 20 | days | 8271 | |
| UR259223918 | uninduced | | 746 | |
| | 1 | day | 26849 | |
| | 13 | days | 22727 | |
| | 20 | days | 8011 | |
| UR259223922 | uninduced | | 48 | |
| | 1 | day | 35542 | |
| | 13 | days | 25491 | |
| | 20 | days | 9388 | |
| UR259223955 | uninduced | | 0 | |
| | 1 | day | 2702 | |
| | 13 | days | 42827 | |
| | 20 | days | 357 | |
| UR259223924 | uninduced | | 294 | Third |
| | 10 | days | 15796 | |
| | 3 | days | 20173 | |
| UR259223927 | uninduced | | 10 | |
| | 10 | days | 21405 | |
| | 10 | days | 38549 | |
| | 3 | days | 32759 | |
| UR259223928 | uninduced | | 80 | |
| | 10 | days | 30073 | |
| | 3 | days | 49933 | |
| UR259223932 | uninduced | | 1 | |
| | 10 | days | 531 | |
| | 3 | days | 739 | |
| UR259223940 | uninduced | | 153 | |
| | 10 | days | 36516 | |
| | 3 | days | 43426 | |
| UR259223945 | uninduced | | 9 | |
| | 10 | days | 35557 | |
| | 3 | days | 49649 | |
| UR259223948 | uninduced | | 55 | |
| | 10 | days | 18665 | |
| | 3 | days | 69260 | |
| UR259223950 | uninduced | | 77 | |
| | 10 | days | 29134 | |
| | 3 | days | 55585 | |
| UR259223952 | uninduced | | 1 | |
| | 10 | days | 1190 | |
| | 3 | days | 1462 | |
| UR259223957 | uninduced | | 266 | |
| | 10 | days | 114840 | |
| | 3 | days | 110737 | |
| UR259223960 | uninduced | | 2 | |
| | 10 | days | 19513 | |
| | 3 | days | 30139 | |
| UR259223902 | uninduced | | 97 | Fourth |
| | 1 | day | 15182 | |
| | 7 | days | 16705 | |
| UR259223904 | uninduced | | 479 | |
| | 1 | day | 7688 | |
| | 7 | days | 3698 | |
| UR259223908 | uninduced | | 18 | |
| | 1 | day | 2927 | |
| | 7 | days | 29994 | |
| UR259223911 | uninduced | | 81 | |
| | 1 | day | 2751 | |
| | 7 | days | 21294 | |
| UR259223912 | uninduced | | 1 | |
| | 1 | day | 467 | |
| | 7 | days | 1243 | |
| UR259223913 | uninduced | | 34 | |
| | 1 | day | 1442 | |
| | 7 | days | 17493 | |
| UR259223914 | uninduced | | 441 | |
| | 1 | day | 20198 | |
| | 7 | days | 39691 | |
| UR259223915 | uninduced | | 168 | |
| | 1 | day | 10521 | |
| | 7 | days | 21298 | |
| UR259223916 | uninduced | | 19 | |
| | 1 | day | 1587 | |
| | 7 | days | 1385 | |
| UR259223919 | uninduced | | 137 | |
| | 1 | day | 22214 | |
| | 7 | days | 50024 | |
| UR259223921 | uninduced | | 8 | |
| | 1 | day | 3198 | |
| | 7 | days | 10167 | |
| UR259223923 | uninduced | | 1 | |
| | 1 | day | 4997 | |
| | 7 | days | 8878 | |
| UR259223925 | uninduced | | 38 | |
| | 1 | day | 5237 | |
| | 7 | days | 12479 | |
| UR259223926 | uninduced | | 9 | |
| | 1 | day | 2924 | |
| | 7 | days | 15035 | |
| UR259223929 | uninduced | | 1501 | |
| | uninduced | | 1275 | |
| | 1 | day | 7260 | |
| | 7 | days | 14542 | |
| UR259223930 | uninduced | | 31 | |
| | 1 | day | 7253 | |
| | 7 | days | 25888 | |
| UR259223931 | uninduced | | 29 | |
| | 1 | day | 1818 | |
| | 7 | days | 11038 | |
| UR259223934 | uninduced | | 0 | |
| | 1 | day | 45 | |
| | 7 | days | 1829 | |
| UR259223935 | uninduced | | 514 | |
| | 1 | day | 22641 | |
| | 7 | days | 37973 | |
| UR259223937 | uninduced | | 105 | |
| | uninduced | | 86 | |
| | 1 | day | 14903 | |
| | 7 | days | 26072 | |
| UR259223938 | uninduced | | 10 | |
| | 1 | day | 910 | |
| | 7 | days | 12048 | |
| UR259223939 | uninduced | | 9 | |
| | 1 | day | 2386 | |
| | 7 | days | 9336 | |
| UR259223941 | uninduced | | 8 | |
| | uninduced | | 6 | |
| | 1 | day | 1179 | |
| | 7 | days | 12298 | |
| UR259223942 | uninduced | | 139 | |
| | 1 | day | 27377 | |
| | 7 | days | 25381 | |
| UR259223943 | uninduced | | 1 | |
| | 1 | day | 16 | |
| | 7 | days | 2460 | |
| UR259223946 | uninduced | | 2 | |
| | 1 | day | 3234 | |
| | 7 | days | 10404 | |
| UR259223947 | uninduced | | 2 | |
| | 1 | day | 57 | |
| | 7 | days | 29 | |
| UR259223949 | uninduced | | 39 | |
| | uninduced | | 46 | |
| | 1 | day | 3496 | |
| | 7 | days | 15911 | |
| UR259223951 | uninduced | | 214 | |
| | uninduced | | 56 | |
| | 1 | day | 8158 | |
| | 7 | days | 24724 | |
| UR259223953 | uninduced | | 34 | |
| | uninduced | | 187 | |
| | 1 | day | 2818 | |
| | 7 | days | 12811 | |
| UR259223954 | uninduced | | 1810 | |
| | uninduced | | 1533 | |
| | 1 | day | 2105 | |
| | 7 | days | 1725 | |
| UR259223965 | uninduced | | 1945 | |
| | uninduced | | 1245 | |
| | 1 | day | 26676 | |
| | 7 | days | 52060 | |

*×1000 relative to endogenous control

We selected a subset of T1 plants from each treatment to score the gl1 mutagenesis rate in the T2 generation. Eight to sixteen siliques from senesced T1 plants were individually collected and all its seeds were sprinkled over an individual 2″×2″ pot. The pots were stratified for four days at 4° C. and then placed in a growth chamber set at 23° C. with 12 hours of light. The leaves were scored for glabrous phenotype by counting the number of seedlings without trichomes and diving them by the total number of seedlings in the pot. A few seedlings were mosaics, with parts of the leaf or leaves being glabrous and parts with trichomes, and we scored those seedlings as glabrous. The average glabrous1 rate was calculated as the mean rate of glabrous seedlings across all pots of the same T1 event.

Table 4, showing the gl1 rate measured for 28 T1 events induced with alcohol at four different times after planting. The rate of gl1 mutagenesis is calculated as the mean rate of gl1 seedlings per pot in the T2 generation (gl1 seedlings/total number of seedlings). Each pot was planted with seed from a unique T1 event silique.

| T1 PlantID | Treatment | Number of pots | mean.gl1 |
|---|---|---|---|
| UR259223961 | First | 16 | 0.027 |
| UR259223920 | | 16 | 0.000 |
| UR259223959 | | 16 | 0.000 |
| UR259223909 | Second | 16 | 0.000 |
| UR259223918 | | 16 | 0.000 |
| UR259223905 | | 16 | 0.002 |
| UR259223906 | | 8 | 0.570 |
| UR259223903 | | 16 | 0.000 |
| UR259223917 | | 16 | 0.000 |
| UR259223922 | | 16 | 0.000 |
| UR259223932 | Third | 8 | 0.000 |
| UR259223952 | | 8 | 0.026 |
| UR259223927 | | 8 | 0.031 |
| UR259223945 | | 8 | 0.062 |
| UR259223950 | | 8 | 0.093 |
| UR259223928 | | 8 | 0.123 |
| UR259223960 | | 8 | 0.172 |
| UR259223957 | | 16 | 0.202 |
| UR259223948 | | 8 | 0.340 |
| UR259223938 | Fourth | 16 | 0.000 |
| UR259223902 | | 24 | 0.000 |
| UR259223911 | | 32 | 0.000 |
| UR259223914 | | 16 | 0.000 |
| UR259223915 | | 16 | 0.000 |
| UR259223919 | | 8 | 0.000 |
| UR259223921 | | 16 | 0.000 |
| UR259223926 | | 16 | 0.000 |
| UR259223953 | | 16 | 0.000 |

To assess the alleles generated by the alcohol-inducible Cas12a we sampled individual T2 gl1 seedlings into 96-well plates for DNA extraction, PCR amplification, and Sanger sequencing of the region around the targeted sequence in gl1. A large number of deletions in gl1 were not expected to results in a glabrous seedling if they are heterozygous because gl1 is recessive; in addition, 3-mer deletions are likely to result in partial to no loss of function. To capture additional alleles of gl1 ‘masked’ by heterozygosity or partial loss of function we also sequenced wild type seedlings in pools of five seedlings. A 614 base pair gl1 fragment was amplified using Q5 DNA polymerase (NEB) with primers GL1_F (CGTGTCACGAAAACCCATC) and GL1_R(TCAACTTAACCGGCCAAATC) and Sanger sequenced with primer GL1_F. The resulting trace chromatograms were analyzed using Synthego's ICE CRISPR analysis tool to infer the nature of the edits (www.biorxiv.org/content/10.1101/251082v3).

Table 5, showing the alignment of gl1 alleles around the target site.

| SEQ | | Base | |
|---|---|---|---|
| ID | | pairs | No. of |
| NO | Sequence | deleted | Samples |
| 11 | ATTCGTTGATAGGGCTAAAGAGATGTGGGAAAAGTTGTAGACTGAGATGGATGAATTAT | wildtype | — |
| 12 | .....................GATG--................................ | 2 bp | 11 |
| 13 | .....................----.................................. | 4 bp | 123 |
| 14 | .....................GA----................................ | 4 bp | 75 |
| 15 | ............-------------.................................. | 13 bp | 12 |
| 16 | .....................GA--..-----........................... | 7 bp | 3 |
| 17 | .....................GA--..----............................ | 6 bp | 1 |
| 18 | .............------------.................................. | 12 bp | 6 |
| 19 | .....................GAT-----.............................. | 5 bp | 3 |
| 20 | .....................GA-----............................... | 5 bp | 15 |
| 21 | .....................-------............................... | 7 bp | 1 |
| 22 | .....................GA------.............................. | 6 bp | 2 |
| 23 | ....................-----------............................ | 11 bp | 1 |
| 24 | .................---------................................. | 9 bp | 15 |
| 25 | ...................-------................................. | 7 bp | 11 |
| 26 | ..................--------................................. | 8 bp | 4 |
| 27 | .....................-----................................. | 5 bp | 15 |
| 28 | .....................GA---................................. | 3 bp | 8 |
| 29 | .....................G----................................. | 4 bp | 81 |
| 30 | ....................------................................. | 6 bp | 3 |
| 31 | ....................-----.................................. | 5 bp | 2 |
| 11 | .....................GATG.................................. | no edits | 157 |
| 32 | ...................-----G.................................. | 5 bp | 48 |
| 33 | .....................---G.................................. | 3 bp | 4 |
| 34 | ...................------.................................. | 6 bp | 41 |
| 35 | .................--------.................................. | 8 bp | 37 |
| 36 | ..............-----------.................................. | 11 bp | 6 |
| 37 | ..................-------.................................. | 7 bp | 38 |
| 38 | ................---------.................................. | 9 bp | 7 |
| 39 | ...............----------.................................. | 10 bp | 4 |
| 40 | ..............------------................................. | 12 bp | 6 |
| 41 | ...............-----------................................. | 11 bp | 1 |
| 42 | .....................GATG-----------....................... | 11 bp | 1 |
| 43 | .................----------................................ | 10 bp | 1 |
| 44 | ..................---------................................ | 9 bp | 1 |
| 45 | ...................--------................................ | 8 bp | 1 |
| 46 | .....................GATG-----............................. | 5 bp | 11 |
| 47 | .....................GAT----............................... | 4 bp | 16 |
| 48 | ..........------------------............................... | 18 bp | 5 |
| 49 | .....................GAT-.................................. | 1 bp | 5 |
| 50 | ..........-------------------------........................ | 25 bp | 2 |
| 51 | ...........-----------------------------................... | 29 bp | 3 |
| 52 | ..........---------------------------...................... | 27 bp | 1 |
| 53 | ..........-----------------------.......................... | 23 bp | 26 |
| 54 | ..........-----------------------------.................... | 29 bp | 8 |
| 55 | ..........------------------------......................... | 24 bp | 1 |
| 56 | ...........-----------------------......................... | 23 bp | 9 |
| 57 | ..........---------------------............................ | 21 bp | 2 |
| 58 | ..........--------------------............................. | 20 bp | 4 |
| 59 | ...........------------------.............................. | 18 bp | 2 |
| 60 | ..........-----------------................................ | 17 bp | 1 |
| 61 | ...........--------------.................................. | 14 bp | 5 |
| 62 | ..........---------------.................................. | 15 bp | 5 |
| 63 | ..........----------------------........................... | 22 bp | 4 |
| 64 | ..........----------------------------..................... | 28 bp | 31 |
| 65 | ..........----------------................................. | 16 bp | 5 |
| 66 | ..........------------------------------................... | 30 bp | 2 |
| 67 | .............-------------------------..................... | 25 bp | 2 |
| 68 | ............---------------------------.................... | 27 bp | 1 |

In Table 5, a partial gl1 sequence is provided as reference. A dot (“.”) indicates the edited sequence possesses an identical nucleotide as the reference sequence (“SEQ ID NO: X”) at that position; likewise, a specified nucleotide (e.g., G, A, T, or C), where provided, also indicates an identical nucleotide as the reference sequence. A dash (“—”) indicates the edited sequence lacks a nucleotide at the corresponding position of the reference sequence. A series of dashes represents the loss of nucleotides equal to the number of dashes. No insertions or substitutions were observed. The column titled “No. Samples” represents the number of DNA samples extracted from T2 individual, or in a few cases pooled, seedlings found to have that edit. Some edits occurred in only one DNA sample; some occurred in several samples. For example, the gl1 sequence in Edit 58 lost twenty-seven base pairs, and only one sample possessed that edit; Edit 2 lost four base pairs and 123 samples were found to have this edit. In total, 277 DNA samples were sequenced, of which 128 were gl1 seedlings, 67 were mosaic, and 52 were wild type. In particular, 96 seedlings were from plate DNA10000156, 55 seedlings were from plate DNA1000104, and 96 seedlings were from plate DNA1000164. After submitting those 277 trace chromatograms to ICE we recovered 896 sequences. Zygosity and bialleleism were not assayed.

Twenty-four plants (Plants 01-24) were in the treatment group (i.e., 2% ethanol drench tray) and eight plants (Plants 33-40) in the control group (i.e., no ethanol). qRT PCR samples were collected at 17 hours, 46 hours, 70 hours and 94 hours.

Seed will be collected from different parts of the Arabidopsis plants inflorescences and planted to evaluate gene editing of the glabrous1 target gene.

Editing will be assessed phenotypically by observation of the presence/absence of trichomes on leaves and by both TaqMan and sequencing of gl1 .

### Example 2. DEX-Induced Mosaicism

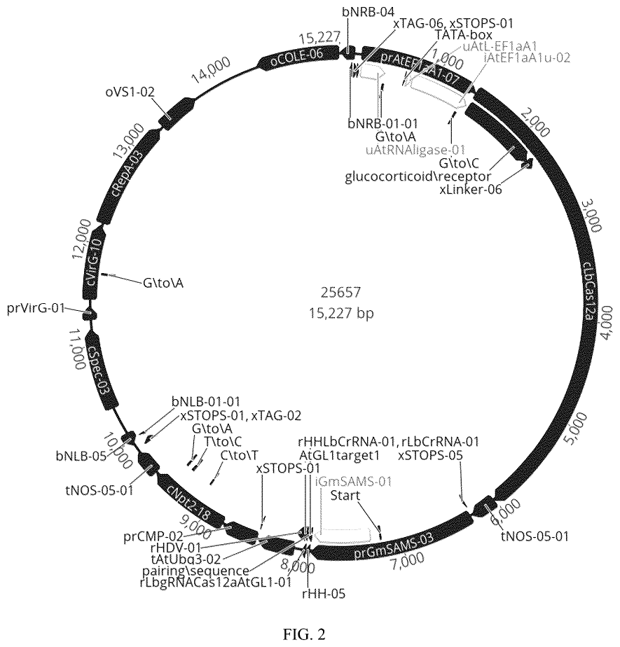

Two vectors for DEX-inducible Cas12a activity were constructed. In the first vector 25657, the glucocorticoid receptor (GR) was fused to Cas12a, driven by a constitutive promoter, prAtEF1aA1-07 (SEQ ID NO:2; FIG. 2). In version two, vector 27057, there is a fusion of Ga14-VP16-GR, driven by pr35S. More details of these two constructs are provided below.

In the first example, the hormone binding domain of the rat glucocorticoid receptor (GR) was fused to an editing enzyme of choice. By fusing the GR domain to Cas12a, this makes its nuclear localization dependent on the application of DEX. Arabidopsis plants were transformed with vector 25657, comprising sequences enabling expression of DEX-inducible Cas12a (“GR-Cas12a”). GR-Cas12a lacks an NLS but comprises a glucocorticoid receptor (GR) binding domain at the N-terminus separated by a long linker. The GR-Cas12 protein is constitutively expressed by the Arabidopsis promoter prAtEF1aA1 but remains localized to the cytoplasm. In the presence of a glucocorticoid, e.g., dexamethasone (“DEX”), the GR-Cas12a translocates to the nucleus. The vector also encodes for a guide RNA targeting 5′-ccacatctctttagccctatcaa-3′ at the second exon of the glabrous 1 (gl1) gene in Arabidopsis.

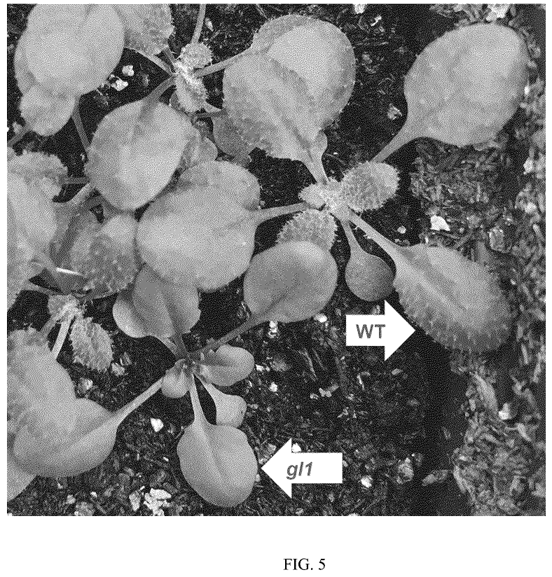

The transformed Arabidopsis plants were grown to the desired developmental stage, e.g., during inflorescent development, at which time a glucocorticoid, e.g., dexamethasone, will be applied to the plants. DEX application may be topically, a root drench, or otherwise. Plants were permitted to develop normally, and progeny will be analyzed for mosaicism. Editing was assessed phenotypically by observation of the presence/absence of trichomes on leaves (see FIG. 5) and by both TaqMan and sequencing of gl1 . However, the 25657 embodiment was found to be not as efficient at editing as desired, either because the version of Cas12a in not sufficiently active or because of other reasons related to the GR-Cas12a fusion. This issue is also reflected in insufficiently high expression of Cas12a.

For vector 27057, the system is based on the interaction properties of a steroid, like dexamethasone, with the recombinant protein GVG, which is composed of yeast (Saccharomyces cerevisiae) GAL4 DNA binding domain, Herpes simplex VP16 activation domain, and the hormone-binding domain from the rat (Rattus norvegicus) glucocorticoid receptor (GR). The hormone-binding domain of the glucocorticoid receptor (“GR”) has a size of 277 amino acids. In the absence of steroids, GVG interacts with cytosolic complexes containing heat shock proteins 90 (“HSP90”) and remains localized to the cytoplasm, making it transcriptionally inactive. After treatment with the synthetic steroid hormone dexamethasone, the GVG/HSP90 interaction is disrupted and the GVG protein localizes to the cell nucleus where it the bind to a regulatory sequence composed of multiple copies of the GAL4 upstream activating sequence (GAL4 UAS). Once bound to this promoter region the VP16 domain activates transcription of the downstream gene. This vector was transformed into Arabidopsis as described above and editing was assessed phenotypically by observation of the presence/absence of trichomes on leaves and by both TaqMan and sequencing of gl1.

### Example 3. Induced Mosaicism

Other inducible systems may be used to obtain induced mosaicism when combined with gene editing technologies and deployed at a desired developmental stage. Usable systems include a galactose-dependent effector (e.g., a VGE inducible system) and a lexA-dependent effector (e.g., a LexA:VP16:ER activator (XVE inducible system)).

In the VGE system, the activator is VP16:Ga14:ER, in the N-terminal to C-terminal direction. In this system, the effector is a promoter comprising at least one but generally four, five, or six Ga14-UAS elements upstream of a minimal promoter.

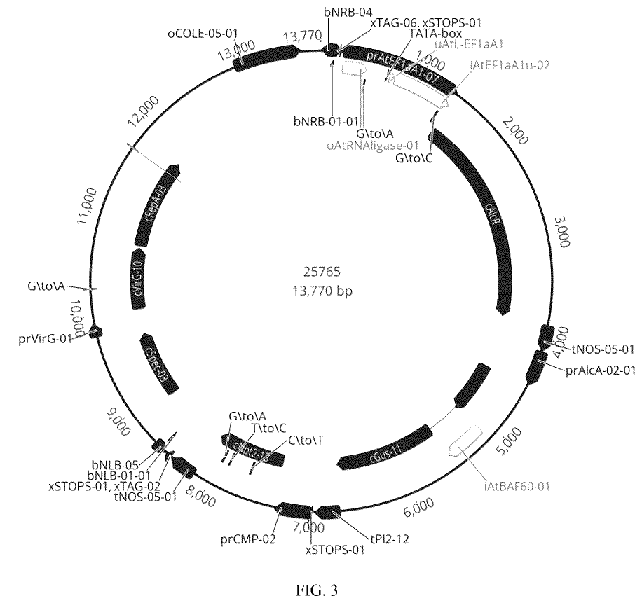

In a lexA-dependent effector-based system, e.g., an XVE inducible system (as described in I. Moore, et al., Transactivated and chemically inducible gene expression in plants, PLANT J., 45:651-683 (2006), at FIG. 3), the activator comprises a Lex:VP16 fusion protein further fused to a steroid receptor, constitutively expressed by, e,g., a 35S promoter. The steroid receptor may be a glucocorticoid receptor (“GR”) or an estradiol receptor (“ER”). In the XVE system, the activator comprises a lexA repressor domain fused to the VP16 transcription activation domain and the human estrogen receptor ER (“Lex:VP16:ER”, or “XVE;” these terms are used interchangeably) in the N-terminal to C-terminal direction. The effector is a promoter comprising at least one but generally four, five, six, seven, or eight lexA operators upstream of a minimal promoter, thus activating the transcription of the gene of interest. In the presence of estrogens like 17-β-estradiol, XVE binds to the multiple copies of the lexA domain, thus activating the transcription of the downstream target—in this case, Cas12a. Alternately, the Cas12a could be constitutively expressed and the XVE activates transcription of a gRNA molecule.

Other systems can be co-opted into application for obtaining inducible mosaicism. See Table 6, below.

Table 6, showing chemically inducible systems usable in plants.

| | Transcription | | |
|---|---|---|---|
| System Type | Factor | Inducer | Reference¹ |
| De-repressible | TetR | Tetracycline | 6 |
| Inactivatable | tTA | Tetracycline | 7 |
| Activatable | GVG | DEX | 10 |
| | AlcR | Ethanol | 14 |
| | | (acetaldehyde) | |
| | GVGEc | RH5992 | 12** |
| | ER-C1 | Beta-estadiol | 11* |
| | XVE | Beta-estadiol | Zuo, |
| | | | unpublished |
| Dual control | TGV | DEX & Tetracycline | 9** |

¹J. Zuo and N.-H. Chua, Chemical-inducible systems for regulated expression of plant genes, CURRENT OP. BIOTECHNOL., 11(2): 146-151, at 157 (2000) (reference numbers in table relate to cited publication).

### REFERENCES

Moore, et al., Transactivated and chemically inducible gene expression in plants, PLANT J., 45:651-683 (2006).

L. Borghi, Inducible Gene Expression Systems for Plants. In: Hennig L., Köhler C. (eds) Plant Developmental Biology. Methods in Molecular Biology (Methods and Protocols), vol 655. Humana Press, Totowa, NJ. doi.org/10.1007/978-1-60761-765-5_5.

Zuo and N.-H. Chua, Chemical-inducible systems for regulated expression of plant genes, CURRENT OP. BIOTECHNOL., 11(2):146-151 (2000).

1. A method for producing a plurality of unique edits in a plant's progeny, comprising:
a. introducing an expression cassette into a plant cell or plant tissue, wherein the expression cassette comprises
i. a nucleic acid encoding a DNA modification enzyme;
ii. an optional nucleic acid encoding at least one guide RNA; and
iii. an inducible factor operably linked to the nucleic acid encoding a DNA modification enzyme;
b. inducing the inducible factor at a desired plant development stage; wherein the inducible factor is a transcription effector or a translocation effector, and
c. generating the plant cell or plant tissue into a plant having progeny, wherein the progeny collectively comprise a plurality of unique edits.

2. (canceled)

3. The method of claim 1, wherein the inducible factor is induced by a chemical selected from an antibiotic, a metal, a steroid, an insecticide, a hormone, an alcohol, and an aldehyde.

4. (canceled)

5. The method of claim 3, wherein the antibiotic is tetracycline or a chemical mimic thereof.

6. The method of claim 3, wherein the metal is copper or a copper-containing compound.

7. The method of claim 3, wherein the steroid is a glucocorticoid is selected from the group consisting of dexamethasone, beclomethasone, betamethasone, budesonide, cortisone, hydrocortisone, methylprednisolone, prednisolone, prednisone, triamcinolone, and any chemical mimic thereof.

8. The method of claim 7, wherein the glucocorticoid is dexamethasone.

9. The method of claim 3, wherein the insecticide is selected from the group consisting of tebufenozide, methoxyfenozide, and any chemical mimic thereof.

10. The method of claim 3, wherein the hormone is selected from the group consisting of estrogen, oestrogen, 17-β-oestradiol, and any chemical mimic thereof.

11. The method of claim 3, wherein the alcohol is selected from the group consisting of ethanol and any chemical mimic thereof.

12. The method of claim 3, wherein the aldehyde is selected from the group consisting of acetaldehyde and any chemical mimic thereof.

13. The method of claim 1, wherein the transcription effector is selected from the group consisting of an alcohol-dependent effector, a lactose-dependent effector, a galactose-dependent effector, and a lexA-dependent effector.

14. The method of claim 13, wherein the alcohol-dependent effector is an an Aspergillus nidulans alc effector comprising an alcA promoter.

15. (canceled)

16. The method of claim 1, further comprising an additional expression cassette comprising a nucleotide sequence encoding an alcR transcription factor activator gene; thereby forming an alcA/alcR inducible system.

17. The method of claim 1, wherein the inducing in step b. comprises applying an alcohol at the desired plant development stage.

18. The method of claim 13, wherein the lactose-dependent effector is a pOp effector.

19. The method of claim 18, further comprising an additional expression cassette comprising a nucleotide sequence encoding a LhG4 transcription factor activator gene; thereby forming an LhG4/pOp inducible system.

20. The method of claim 13, wherein the galactose-dependent regulon is a Gal4 UAS effector.

21. The method of claim 20, further comprising an additional expression cassette comprising a nucleotide sequence encoding a Gal4 transcription factor activator gene; thereby forming a GVG inducible system or a VGE inducible system.

22. The method of claim 13, wherein the lexA-dependent effector is at least one LexA operon.

23. The method of claim 22, further comprising an additional expression cassette comprising a nucleotide sequence encoding a LexA:VP16:ER activator; thereby forming an XVE inducible system.

24. The method of claim 1, wherein the DNA modification enzyme is selected from the group consisting of a meganuclease (MN), a zinc-finger nuclease (ZFN), a transcription-activator like effector nuclease (TALEN), a chimeric FEN1-FokI, a Mega-TALs, and a CRISPR nuclease.

25. The method of claim 24, wherein the CRISPR nuclease is a Cas nuclease, a Cas9 nuclease, a Cpf1 nuclease, a dCas9-FokI, a dCpf1-FokI, a chimeric Cas9-cytidine deaminase, a chimeric Cas9-adenine deaminase, a nickase Cas9 (nCas9), a chimeric dCas9 non-FokI nuclease, and a dCpf1 non-FokI nuclease, a Cas12a fused to a deaminase domain, a Cas12i nuclease, a Cas12j nuclease, a CasX nuclease, a CasY nuclease, a Cas13 nuclease, a Cas14 nuclease.

26. The method of claim 1, wherein the translocation factor is a glucocorticoid receptor comprising SEQ ID NO: 6.

27. (canceled)

28. The method of claim 26, wherein the glucocorticoid receptor is operably linked to a modified Cas12a nuclease modified to comprise a glucocorticoid receptor binding domain (“GR-Cas12a”) comprising SEQ ID NO: 7.

29. (canceled)

30. (canceled)

31. The method of claim 28, wherein upon application of dexamethasone, the GR-Cas12a translocates from the cytoplasm to the nucleus of the plant cell or plant tissue.

32. The method of claim 1, wherein the unique edit is an indel mutation, a nucleotide substitution, an allele replacement, a chromosomal translocation, or an insertion of donor nucleic acid.

33. The method of claim 1, wherein the plant cell or plant tissue is dicotyledonous selected from the group consisting of Arabidopsis, sunflower, soybean, tomato, Brassica species, Populus (poplar), Eucalyptus, tobacco, Cannabis, potato, cotton, maize, rice, wheat, barley, sugarcane, Glycine tomentella, and other wild Glycine species.

34. (canceled)

35. The method of claim 1, wherein the plant cell or plant tissue is monocotyledonous selected from the group consisting of maize, wheat, rice, teosinte, sorghum, and barley.

36. (canceled)

37. The method of claim 35, wherein the monocotyledonous plant cell or plant tissue is maize and wherein the desired developmental stage is selected from the group consisting of VE, V1, V2, V(n), VT, R1, R2, R3, R4, R5, and R6 stage; where (n) is an integer representing the number of leaf collars present.

38. (canceled)

39. The method of claim 33, wherein plant cell or plant tissue is soybean and wherein the desired developmental stage is selected from the group consisting of VE, VC, V1, V2, V(n), R1, R2, R3, R4, R5, R6, R7, and R8 stage; where (n) is an integer representing the number of trifoliolates present.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)


SEQUENCE LISTING (69 sequences)

SEQ ID NO: 1 - DNA, 13484 bp, Artificial Sequence
  Vector 24902
  promoter (521)..(1045): 35S promoter
  gene (1071)..(2384): cGal4VP16GR-n1 GAL4-binding domain-VP16 activation domain-GR (for glucocorticoid receptor) fusion (GVG)
  terminator (2396)..(3038): PsE9 terminator
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccgagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagtttaaacaatggcagatccaatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctaggtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccaattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgacttgagctcttagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaataacctgcaggtcgaagcttgcatgccggtcgactctagaggatccgggtgacagccctccgacgggtgacagccctccgacgggtgacagccctccgaattctagaggatccgggtgacagccctccgacgggtgacagccctccgacgggtgacagccctccgaattcgagctcggtacccggggatctgtcgacctcgatcgagatcttcgcaagacccttcctctatataaggaagttcatttcatttggagaggacacgctgaagctagtcgactctagcctgcgatcgctaaaaaatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagtaacgcgtttatgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaccgctaattagctaagagacacttgtgtgattgagagaaacactaatcttgtgaggactgaagtttggtgattatttcttgtgatctgtcgacaaaaatatcaaatggggtttcttttacaaattatttacctaaatgaatctgttttgaaaatatttactccattgggtctatttttttattacaaagcgtctccctgaagggcgcgttccccgtgaaagtgacacgtggcaggacttgggacgtgccctgcgtacaggcgcgatagttagtgttgttacagcaggcgcatcgggtcgtgttggggaccaaggtacgacaggtcgcgctgggtgacccagacacgacccaattgggtcgcactttatttaatattttttatattttgtatattgtttttatttaatatatttttatattattttatttaatttttttatattttatataatagtttctatattaaataaattcttagcattatgtatgattttaaagtcataaataattttttatattgtttttatttactatattttttatattttatttaatatttatatattaaataaatccttcatattagaaaaaataaagaaaatattaaataaaatataaaatataaaaaagtaaaaaatattaaataaaataatataaaaaatattataaaaacaatataaaaaatataaaaatatttaataaaataataaaaaaaatattattttaaataaaattatttatgactttaaactctaaagttgaattttaaaaaaatataatttttttacgattttagtaaaaaaaaaatacaagccgcacaatacaagtcgccttctcaaacccttcctcacgacattctcggaccttatgacaccgtcaccaaaacaatgatccacgcgatattaggcgcgtgcaaatcactctaatccgaaactagtagacatgggaagcacgagctatacgcgagcgtttcaattgccgccacgaaagcagagaaggccagaaacggaaccacggtaaaatggtaagggtattttcgtaaacagaagaaaagagttgtagctataaataaaccctctaacccacggcgcactatttctcttcactccttcgttcactcttcttctcttgcggctagggttttagcgcagcttcttctaggttcgttctcttccgccgctctatggattttaaaccttcgaatcatgtttattccattgaattatgttgcttgcagtttatattttctgaatctgtagttgttgtcttcaatttatcctatgctttatagatcaatcttttgtgtgtgtagtacgtaatttttgttctttttgcttttcgttcaagttgttgggaataatcggggtatcatgttttgatattgtttgttttcttttttgactgcttaataatttttaagttggttttggttttggggttttatgtgcttgttatattcaaatctttgtgatccagatcttacaaaagttttgggtttaaggatgtttttggctgatgatgaatagatctataaactgttccttttaatcgattcaagcttaggattttactaggcttttgcgaataaatacgtgacagtaagctaattatgtcctttttttgtctcaatcatatctgtctgggtgtgccataatttgtgatatgtctatctggtagaatcttgtgttttatgctttacgatttggtatacctgtttttgaacttgttgtatgatgggtatttagatcaccctatcttttttatgcttctggaagttttatgtaaatgtcgaatatcttaatgttgttgaacttataatgttgtgttgatgtatgtatgatggttttgacaacttttttcactggttctgaaagttttatgtaaattgcaaatatgttaatgttgttgaacttattttttttccttcgatgttgttttgatgtatgtatgatggttttcaccgtagtttctatggctaatatcttaatgttgttgagcttatttttttccttatatgttgtgttgatgtattgtatgatggttttgacaacttttttagtttctttgcagatttaaggaagaaaaaaaaaatggctcttgttgctagaccagtgctttctgctagagttgctgcttctagaccaagagttgctgcaagaaaggctgttagagtgtctgctatgtctcatggtgcttcatctagaccagctactgctagaaagtcctctggactttctggaactgttaggattccaggcgataagtctatttcccaccgttctttcatgttcggaggacttgcttctggtgagactagaattactggacttcttgagggcgaggatgttattaacactggaaaggctatgcaggctatgggagctagaattagaaaagagggcgatacctggattattgatggtgttggaaacggtggacttcttgctccagaagctccacttgatttcggaaacgctgctactggatgcagacttactatgggacttgttggagtgtacgatttcgattccaccttcattggagatgcttctcttactaagaggccaatgggaagagtgcttaacccacttagagagatgggagttcaggttaagtctgaggatggtgataggcttccagttactcttagaggaccaaagactccaaccccaattacttacagagtgccaatggcttctgctcaagttaagtctgctgttcttcttgctggacttaacactccaggtattaccactgtgatcgagccaattatgaccagagatcataccgagaagatgcttcaaggattcggagctaaccttaccgttgaaactgatgctgatggtgttaggaccattagacttgagggaagaggaaagcttaccggacaagttattgatgttccaggtgatccatcttctaccgctttccctcttgttgctgctcttttggttccaggatctgatgtgaccattcttaacgtgctcatgaacccatctaggaccggacttattcttacccttcaagaaatgggcgctgatattgaggttatcaacccaagacttgctggtggtgaagatgttgctgatcttagggttaggtcctctactcttaagggtgttactgttccagaagatagggctccatccatgattgatgagtacccaattcttgctgtggctgctgctttcgctgaaggtgctactgttatgaacggacttgaggaacttagggtgaaagagtctgataggctttctgctgttgctaacggacttaagcttaacggtgtggattgtgatgagggtgagacttctcttgttgttagaggtagaccagatggaaagggacttggaaacgcttctggtgcagcagttgcaactcatcttgatcataggatcgctatgtccttccttgttatgggtcttgtttctgagaacccagttaccgttgatgatgctactatgattgctaccagcttcccagagttcatggatcttatggctggacttggagctaagattgagctttctgataccaaggctgcctgactaattagctaagagcttgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 2 - DNA, 15227 bp, Artificial Sequence
  Vector 25657
  promoter (217)..(1376): AtEF1aA1 promoter
  gene (1395)..(6008): GR-LbCas12a  Rice codon-optimized cLbCas12a-02 without NLS, adding a glucocorticoid receptor (GR) binding domain at the N-terminus separated by a long linker.
  terminator (6017)..(6269): tNOS
  promoter (6288)..(7901): prGmSAMS
  misc_feature (7914)..(7950): Hammerhead ribozyme
  misc_feature (7951)..(7994): LbCas12a guide RNA targeting AtGL1
  misc_feature (7995)..(8062): HDV ribozyme
  terminator (8063)..(8408): tAtUbq3
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttggaagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatctctactgtttttattgattcaatatttgattgttcttttttttgcagatttgttgaccagggatcccctaaatagaccatggctattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaagggggcgggggctcaggcgggggcgggagcggcggcgggggctctgggggcggcggcagcggcgggggcggcagcgggggcggcgggtcgatgtcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgagggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcaaaaaagttcatatctagttttgatcgtattatgtacgtcccagaggaagatcttttcgagtttgctctcgactacaaaaacttttcgcgcaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcattgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaattaactagctagtccaccctcacaaaaaaggttattgcctaattaatagtagggattgaagaagtatcaataatcaataagagggacatttggattgacacaaactattagtataaaaaaaaaatgtaaaactaaatggacaacaatctaagaaagcgagtatgtgttgtactcggaaaacataacataatgtgattttgattgtggaaactgaaaacaataacaattaagttttatttatctctttgcctaacaatttttttaattactttttttatagtttttatgatgaagataattaatattttttacaaatattatattttcctttttaccatctttgaagataattgcttttttttccaatttagacaaatatttgagtatcaatgatattcctttttaatccattattgcaatttaaaagaccatcaatgattacattttcaatcaaatgactttattagtcttcactttacatcgatttaaatcaaactaataattttgtatggactaaatctctgaacatttttatatttacaacatattttaacatttattaaataattagtatttaatactattagtagaataatgggagtagcaggagggaggcactgagagaatagagatggcatggaagtaagcaatcaagtcaaaatcagagttggccaaccccaaaggctgtagtaggtaagcatggcccattttagtttttacattcatctctcattttcacctcaacggttcagattcaatctgactccccgatctcagccgtggattcaaatgccacctcaggcacatgcaattccaaatggatgaacctaacccacaatctaatcttgttacttaggggcttttccgtcattaaatgacaccacctacccccttctccctataaatggcaactcaatcccccccttagaactcgcagcgcttgatttgaggccaggcaagccccactcaaccaccacacctctcctcgttcacgctacccctttctgctcttcttctacctttcaaggtactcttctttccctctgttgctgcaaccttctctttctttaagattgcctcaatttcggatcttgcacctctgggttgctttgctttgctttttcctctactgggttgatttctgtttccctaaaccggtttagacgaatgtgaacactacttcttttgtttaattactctggaatacgtgttaggctttcagatctagttgaaatcgtattgcacttttagggggagtttggatttctaataagaaattgaccttttgctgagaattggttcggtgattagagggtttccgtaaatttttgaagttttacatgcttgtatctgtttatttttgtttctcacatctattattgttaggtgaaggaaattatgtattgagagtctgtctgatactaaatataaacacctcaataggggctctaacactgattttatcatttgctgcttgtgtgtatggttaaagaaaggcaattgtgttttaattttctgcaagctttcgtttgctgaattttatgcatatattttcctcccttttgtgaacttcctttttgtagttctaattccatttttggtgtctgcagttttaaaagtataaagggatccaaattactgatgagtccgtgaggacgaaacgagtaagctcgtctaatttctactaagtgtagatccacatctctttagccctatcaaggccggcatggtcccagcctcctcgctggcgccggctgggcaacatgcttcggcatggcgaatgggacagctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaacttgaatgttcactactacggaccgctagtgcatgcactgactaattagctaactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaagacctaaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccacggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcattagcctgcaggcccgggttagtctaattagctaacggccaggatcgccgcgtgagcctttagcaactagctagagcgatcgccgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacagataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 3 - DNA, 13770 bp, Artificial sequence
  Vector 25765
  promoter (217)..(1376): prAtEF1aA1
  gene (1395)..(3857): AlcR Arabidopsis-optimized alcohol receptor gene (AlcR) from Aspergillus nidulans
  terminator (3869)..(4121): tNOS
  promoter (4128)..(4463): prAlcA
  exon (4482)..(5096): Gus
  Intron (5097)..(5505): iAtBAF60
  exon (5506)..(6702): Gus
  terminator (6711)..(6950): tPI2
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttggaagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatctctactgtttttattgattcaatatttgattgttcttttttttgcagatttgttgaccagggatcccctaaatagaccatggctgatactcgaagaagacagaatcattcttgtgatccatgccgcaaaggtaaacgtcggtgtgacgcacctgaaaacagaaatgaagcaaacgagaatggatgggtgagctgttcaaactgtaagagatggaacaaagattgcactttcaactggttatcgtcccagagaagcaaggcgaaaggtgcagctccacgggctcgaaccaagaaagctagaactgccaccacgacatctgaaccatcaacatctgcagcaaccattccaacaccagagtcggacaaccatgacgctcctccggtgataaactctcacgatgccctccctagttggacccagggactattatcgcatcctggggacctttttgacttttctcatagtgccattccggcgaatgcagaagatgcagccaatgttcaatcggatgctccatttccatgggatcttgcaattccaggtgatttctcaatgggacagcagcttgagaaacctctctcccctttgtcattccaagctgtcctattacctcctcattccccaaacacagatgatcttataagagagctagaagagcaaaccactgatcctgattcagttacggacacaaacagtgtgcaacaagttgctcaagatggatccttgtggtcagacaggcaaagtccgttactacctgaaaattctctctgcatggcttcggactcaaccgctcgtagatatgcacggagtacaatgacaaaaaacttgatgcgtatctaccatgattccatggagaacgctctttcttgctggctcactgaacacaattgtccttattctgatcaaatctcttatctcccacctaagcaaagggctgaatggggtcctaattggagcaataggatgtgtataagggtttgtagacttgatcgtgtaagtacgtcactgagaggcagagcattatcagcagaagaagacaaagctgctgctagagcccttcatctggcgattgttgcattcgcatctcaatggacgcaacacgctcaacgcggagctggtttgaatgtcccggctgacattgctgccgacgaaagatcaatcaggagaaacgcttggaatgaggcaagacacgcgttgcagcatactactgggatcccttctttccgagtgattttcgctaatatcatcttcagcctgactcagagtgtactggatgatgatgaacaacatggaatgggtgcgcgattggacaagcttttggagaatgatggtgcaccggttttcttggaaactgctaataggcaattgtacacatttcgtcacaagtttgccagaatgcaacggagaggcaaggcttttaatcgcttacccggtggttccgtcgcttccacttttgctggaatatttgagaccccaactccatctagtgagtctcctcaacttgatccagttgttgcatctgaggaacatcgtagcactctatcgctcatgttctggcttggaataatgtttgatacactttcagctgcaatgtaccagagaccattagtggtatctgatgaggattcacagatatccagtgcttctcccccgagacgaggcgccgagactcctatcaatttagactgttgggaacctccacgtcaagtcccttcgaatcaagaaaagtctgatgtttggggagatttgtttctcagaacatcagactctttaccagatcatgaatcacacacacagatttctcagccagctgcaaggtggccttgcacctatgagcaggctgcggccgcattgagctcagcgacacctgttaaggttttgctctacaggagagtgactcaactgcaaacacttctatacaggggggcgtctccagcgaggcttgaagctgcgatccaaagaaccctttacgtgtataatcactggacagctaagtatcaacctttcatgcaggattgtgttgcaaatcatgagttgttgcccagccggattcagtcatggtacgtcatccttgacggccattggcatcttgctgctatgttgcttgctgatgtgttagaaagcatagatagagattcgtatagcgatataaaccacattgatttggttacgaagttgcgtctcgataatgctctagccgtctctgcccttgcgagatcttccttgcgtggtcaagagcttgatccggggaaagcatcaccaatgtataggcatttccatgatagtctaacggaagtggcttttcttgtcgagccgtggactgtagtacttattcactcttttgcaaaggcagcttatattctcttagattgtttggatctcgacggacaaggaaacgcactggcgggctacctgcagctccgtcagaactgcaactattgcatccgagctcttcagtttttaggtagaaaatccgatatggcagctctagtagctaaagacctcgagaggggtttaaacggaaaagttgatagctttctgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctccggatcgatagttgtgatagttcccacttgtccgtccgcatcggcatccgcagctcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataaagacccccaaggttctcagtctcaccaacatcatcaaccggatcccctaaatagaccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgattaattaagaccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatataccggaccgctagtgcatgcactgactaattagctaactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaagacctaaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccacggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcattagcctgcaggcccgggttagtctaattagctaacggccaggatcgccgcgtgagcctttagcaactagctagagcgatcgccgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacagataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 4 - DNA, 17492 bp, Artificial sequence
  Vector 25881
  promoter (217)..(1376): prAtEF1aA1
  gene (1395)..(3860): AlcR transcription factor from Aspergillus nidulans optimized for expression in dicots.
  terminator (3869)..(4121): tNOS
  promoter (4128)..(4463): prAlcA
  gene (4482)..(8264): LbCas12a This Cas12a is a rice codon-optimized version from Lachnospiraceae bacterium ND2006
  terminator (8272)..(8511): tPI2
  promoter (8550)..(10163): prGmSAMS
  misc_feature (10176)..(10212): Hammerhead ribozyme
  misc_feature (10213)..(10256): LbCas12a guide RNA targeting AtGl1
  misc_feature (10257)..(10324): HDV ribozyme
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttggaagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatctctactgtttttattgattcaatatttgattgttcttttttttgcagatttgttgaccagggatcccctaaatagaccatggctgatactcgaagaagacagaatcattcttgtgatccatgccgcaaaggtaaacgtcggtgtgacgcacctgaaaacagaaatgaagcaaacgagaatggatgggtgagctgttcaaactgtaagagatggaacaaagattgcactttcaactggttatcgtcccagagaagcaaggcgaaaggtgcagctccacgggctcgaaccaagaaagctagaactgccaccacgacatctgaaccatcaacatctgcagcaaccattccaacaccagagtcggacaaccatgacgctcctccggtgataaactctcacgatgccctccctagttggacccagggactattatcgcatcctggggacctttttgacttttctcatagtgccattccggcgaatgcagaagatgcagccaatgttcaatcggatgctccatttccatgggatcttgcaattccaggtgatttctcaatgggacagcagcttgagaaacctctctcccctttgtcattccaagctgtcctattacctcctcattccccaaacacagatgatcttataagagagctagaagagcaaaccactgatcctgattcagttacggacacaaacagtgtgcaacaagttgctcaagatggatccttgtggtcagacaggcaaagtccgttactacctgaaaattctctctgcatggcttcggactcaaccgctcgtagatatgcacggagtacaatgacaaaaaacttgatgcgtatctaccatgattccatggagaacgctctttcttgctggctcactgaacacaattgtccttattctgatcaaatctcttatctcccacctaagcaaagggctgaatggggtcctaattggagcaataggatgtgtataagggtttgtagacttgatcgtgtaagtacgtcactgagaggcagagcattatcagcagaagaagacaaagctgctgctagagcccttcatctggcgattgttgcattcgcatctcaatggacgcaacacgctcaacgcggagctggtttgaatgtcccggctgacattgctgccgacgaaagatcaatcaggagaaacgcttggaatgaggcaagacacgcgttgcagcatactactgggatcccttctttccgagtgattttcgctaatatcatcttcagcctgactcagagtgtactggatgatgatgaacaacatggaatgggtgcgcgattggacaagcttttggagaatgatggtgcaccggttttcttggaaactgctaataggcaattgtacacatttcgtcacaagtttgccagaatgcaacggagaggcaaggcttttaatcgcttacccggtggttccgtcgcttccacttttgctggaatatttgagaccccaactccatctagtgagtctcctcaacttgatccagttgttgcatctgaggaacatcgtagcactctatcgctcatgttctggcttggaataatgtttgatacactttcagctgcaatgtaccagagaccattagtggtatctgatgaggattcacagatatccagtgcttctcccccgagacgaggcgccgagactcctatcaatttagactgttgggaacctccacgtcaagtcccttcgaatcaagaaaagtctgatgtttggggagatttgtttctcagaacatcagactctttaccagatcatgaatcacacacacagatttctcagccagctgcaaggtggccttgcacctatgagcaggctgcggccgcattgagctcagcgacacctgttaaggttttgctctacaggagagtgactcaactgcaaacacttctatacaggggggcgtctccagcgaggcttgaagctgcgatccaaagaaccctttacgtgtataatcactggacagctaagtatcaacctttcatgcaggattgtgttgcaaatcatgagttgttgcccagccggattcagtcatggtacgtcatccttgacggccattggcatcttgctgctatgttgcttgctgatgtgttagaaagcatagatagagattcgtatagcgatataaaccacattgatttggttacgaagttgcgtctcgataatgctctagccgtctctgcccttgcgagatcttccttgcgtggtcaagagcttgatccggggaaagcatcaccaatgtataggcatttccatgatagtctaacggaagtggcttttcttgtcgagccgtggactgtagtacttattcactcttttgcaaaggcagcttatattctcttagattgtttggatctcgacggacaaggaaacgcactggcgggctacctgcagctccgtcagaactgcaactattgcatccgagctcttcagtttttaggtagaaaatccgatatggcagctctagtagctaaagacctcgagaggggtttaaacggaaaagttgatagctttctgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctccggatcgatagttgtgatagttcccacttgtccgtccgcatcggcatccgcagctcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataaagacccccaaggttctcagtctcaccaacatcatcaaccggatcccctaaatagaccatggctcctaagaagaagcggaaggttggtattcacggggtgcctgcggcttcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgagggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcaaaaaagttcatatctagttttgatcgtattatgtacgtcccagaggaagatcttttcgagtttgctctcgactacaaaaacttttcgcgcaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcataagcgtcctgctgccaccaaaaaggccggacaggctaagaaaaagaagtgattaatcggaccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatataccggaccgaatctagattcagaacgaattaactagctagtccaccctcacaaaaaaggttattgcctaattaatagtagggattgaagaagtatcaataatcaataagagggacatttggattgacacaaactattagtataaaaaaaaaatgtaaaactaaatggacaacaatctaagaaagcgagtatgtgttgtactcggaaaacataacataatgtgattttgattgtggaaactgaaaacaataacaattaagttttatttatctctttgcctaacaatttttttaattactttttttatagtttttatgatgaagataattaatattttttacaaatattatattttcctttttaccatctttgaagataattgcttttttttccaatttagacaaatatttgagtatcaatgatattcctttttaatccattattgcaatttaaaagaccatcaatgattacattttcaatcaaatgactttattagtcttcactttacatcgatttaaatcaaactaataattttgtatggactaaatctctgaacatttttatatttacaacatattttaacatttattaaataattagtatttaatactattagtagaataatgggagtagcaggagggaggcactgagagaatagagatggcatggaagtaagcaatcaagtcaaaatcagagttggccaaccccaaaggctgtagtaggtaagcatggcccattttagtttttacattcatctctcattttcacctcaacggttcagattcaatctgactccccgatctcagccgtggattcaaatgccacctcaggcacatgcaattccaaatggatgaacctaacccacaatctaatcttgttacttaggggcttttccgtcattaaatgacaccacctacccccttctccctataaatggcaactcaatcccccccttagaactcgcagcgcttgatttgaggccaggcaagccccactcaaccaccacacctctcctcgttcacgctacccctttctgctcttcttctacctttcaaggtactcttctttccctctgttgctgcaaccttctctttctttaagattgcctcaatttcggatcttgcacctctgggttgctttgctttgctttttcctctactgggttgatttctgtttccctaaaccggtttagacgaatgtgaacactacttcttttgtttaattactctggaatacgtgttaggctttcagatctagttgaaatcgtattgcacttttagggggagtttggatttctaataagaaattgaccttttgctgagaattggttcggtgattagagggtttccgtaaatttttgaagttttacatgcttgtatctgtttatttttgtttctcacatctattattgttaggtgaaggaaattatgtattgagagtctgtctgatactaaatataaacacctcaataggggctctaacactgattttatcatttgctgcttgtgtgtatggttaaagaaaggcaattgtgttttaattttctgcaagctttcgtttgctgaattttatgcatatattttcctcccttttgtgaacttcctttttgtagttctaattccatttttggtgtctgcagttttaaaagtataaagggatccaaattactgatgagtccgtgaggacgaaacgagtaagctcgtctaatttctactaagtgtagatccacatctctttagccctatcaaggccggcatggtcccagcctcctcgctggcgccggctgggcaacatgcttcggcatggcgaatgggacagctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaacttgaatgttcactactacggacgaccgctagtgcatgcactgactaattagctaactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaagacctaaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccacggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcattagcctgcaggcccgggttagtctaattagctaacggccaggatcgccgcgtgagcctttagcaactagctagagcgatcgccgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacagataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 5 - protein, 437 aa, Artificial Sequence
  Gal4VP16GR
MADPMKLLSSIEQACDICRLKKLKCSKEKPKCAKCLKNNWECRYSPKTKRSPLTRAHLTEVESRLERLEQLFLLIFPRSSAPPTDVSLGDELHLDGEDVAMAHADALDDFDLDMLGDGDSPGPGFTPHDSAPYGALDMADFEFEQMFTDALGIDEYGGDPIQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQK

SEQ ID NO: 6 - protein, 277 aa, Rattus norvegicus
MQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQK

SEQ ID NO: 7 - protein, 1537 aa, artificial sequence
  GR-Cas12a
MAIQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQKGGGGSGGGGSGGGGSGGGGSGGGGSGGGGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 8 - DNA, 614 bp, artificial sequence
  614 bp gl1 fragment amplicon
cgtgtcacgaaaacccatcataagttcactactcataatctctttctctctctcacacacacacacagacacacacaaacatagaaggaatcagagaataaaaaaagaatgagaataaggagaagagatgaaaaagagaatcaagaatacaagaaaggtttatggacagttgaagaagacaacatccttatggactatgttcttaatcatggcactggccaatggaaccgcatcgtcagaaaaactggtactctttctctctctcgcgcgttttgtgattatatatattaactttagtctctagctagttcatatataaatatatctcagttagactaaatttgattcgttgatagggctaaagagatgtgggaaaagttgtagactgagatggatgaattatttgagccctaatgtgaacaaaggcaatttcactgaacaagaagaagacctcattattcgtctccacaagctcctcggcaataggtacataactttatgttactacaatattatacaccatgtctttttatttaatcgatcgttaactgattttttctttcgtcaaatatttgaattgttgttttaaacaaagatttggccggttaagttga

SEQ ID NO: 9 - DNA, 19 bp, Artificial Sequence
  gl1 forward primer
cgtgtcacgaaaacccatc

SEQ ID NO: 10 - DNA, 20 bp, Artificial Sequence
  gl1 reverse primer
tcaacttaaccggccaaatc

SEQ ID NO: 11 - DNA, 59 bp, Arabidopsis thaliana
attcgttgatagggctaaagagatgtgggaaaagttgtagactgagatggatgaattat

SEQ ID NO: 12 - DNA, 57 bp, Arabidopsis thaliana
attcgttgatagggctaaagagatgggaaaagttgtagactgagatggatgaattat

SEQ ID NO: 13 - DNA, 55 bp, Arabidopsis thaliana
attcgttgatagggctaaagatgggaaaagttgtagactgagatggatgaattat

SEQ ID NO: 14 - DNA, 55 bp, Arabidopsis thaliana
attcgttgatagggctaaagagaggaaaagttgtagactgagatggatgaattat

SEQ ID NO: 15 - DNA, 46 bp, Arabidopsis thaliana
attcgttgatagtgggaaaagttgtagactgagatggatgaattat

SEQ ID NO: 16 - DNA, 52 bp, Arabidopsis thaliana
attcgttgatagggctaaagagatgagttgtagactgagatggatgaattat

SEQ ID NO: 17 - DNA, 53 bp, Arabidopsis thaliana
attcgttgatagggctaaagagatgaagttgtagactgagatggatgaattat

SEQ ID NO: 18 - DNA, 47 bp, Arabidopsis thaliana
attcgttgataggtgggaaaagttgtagactgagatggatgaattat

SEQ ID NO: 19 - DNA, 54 bp, Arabidopsis thaliana
attcgttgatagggctaaagagataaaagttgtagactgagatggatgaattat

SEQ ID NO: 20 - DNA, 54 bp, Arabidopsis thaliana
attcgttgatagggctaaagagagaaaagttgtagactgagatggatgaattat

SEQ ID NO: 21 - DNA, 52 bp, Arabidopsis thaliana
attcgttgatagggctaaagagaaaagttgtagactgagatggatgaattat

SEQ ID NO: 22 - DNA, 53 bp, Arabidopsis thaliana
attcgttgatagggctaaagagaaaaagttgtagactgagatggatgaattat

SEQ ID NO: 23 - DNA, 48 bp, Arabidopsis thaliana
attcgttgatagggctaaagaagttgtagactgagatggatgaattat

SEQ ID NO: 24 - DNA, 50 bp, Arabidopsis thaliana
attcgttgatagggctagggaaaagttgtagactgagatggatgaattat

SEQ ID NO: 25 - DNA, 52 bp, Arabidopsis thaliana
attcgttgatagggctaaagggaaaagttgtagactgagatggatgaattat

SEQ ID NO: 26 - DNA, 51 bp, Arabidopsis thaliana
attcgttgatagggctaagggaaaagttgtagactgagatggatgaattat

SEQ ID NO: 27 - DNA, 54 bp, Arabidopsis thaliana
attcgttgatagggctaaagagggaaaagttgtagactgagatggatgaattat

SEQ ID NO: 28 - DNA, 56 bp, Arabidopsis thaliana
attcgttgatagggctaaagagagggaaaagttgtagactgagatggatgaattat

SEQ ID NO: 29 - DNA, 55 bp, Arabidopsis thaliana
attcgttgatagggctaaagaggggaaaagttgtagactgagatggatgaattat

SEQ ID NO: 30 - DNA, 53 bp, Arabidopsis thaliana
attcgttgatagggctaaaggggaaaagttgtagactgagatggatgaattat

SEQ ID NO: 31 - DNA, 54 bp, Arabidopsis thaliana
attcgttgatagggctaaagtgggaaaagttgtagactgagatggatgaattat

SEQ ID NO: 32 - DNA, 54 bp, Arabidopsis thaliana; identical to SEQ ID NO: 31

SEQ ID NO: 33 - DNA, 56 bp, Arabidopsis thaliana
attcgttgatagggctaaagagtgggaaaagttgtagactgagatggatgaattat

SEQ ID NO: 34 - DNA, 53 bp, Arabidopsis thaliana
attcgttgatagggctaaatgggaaaagttgtagactgagatggatgaattat

SEQ ID NO: 35 - DNA, 51 bp, Arabidopsis thaliana
attcgttgatagggctatgggaaaagttgtagactgagatggatgaattat

SEQ ID NO: 36 - DNA, 48 bp, Arabidopsis thaliana
attcgttgatagggtgggaaaagttgtagactgagatggatgaattat

SEQ ID NO: 37 - DNA, 52 bp, Arabidopsis thaliana
attcgttgatagggctaatgggaaaagttgtagactgagatggatgaattat

SEQ ID NO: 38 - DNA, 50 bp, Arabidopsis thaliana
attcgttgatagggcttgggaaaagttgtagactgagatggatgaattat

SEQ ID NO: 39 - DNA, 49 bp, Arabidopsis thaliana
attcgttgatagggctgggaaaagttgtagactgagatggatgaattat

SEQ ID NO: 40 - DNA, 47 bp, Arabidopsis thaliana
attcgttgataggggggaaaagttgtagactgagatggatgaattat

SEQ ID NO: 41 - DNA, 48 bp, Arabidopsis thaliana
attcgttgatagggcgggaaaagttgtagactgagatggatgaattat

SEQ ID NO: 42 - DNA, 48 bp, Arabidopsis thaliana
attcgttgatagggctaaagagatggtagactgagatggatgaattat

SEQ ID NO: 43 - DNA, 49 bp, Arabidopsis thaliana
attcgttgatagggctaggaaaagttgtagactgagatggatgaattat

SEQ ID NO: 44 - DNA, 50 bp, Arabidopsis thaliana
attcgttgatagggctaaggaaaagttgtagactgagatggatgaattat

SEQ ID NO: 45 - DNA, 51 bp, Arabidopsis thaliana
attcgttgatagggctaaaggaaaagttgtagactgagatggatgaattat

SEQ ID NO: 46 - DNA, 54 bp, Arabidopsis thaliana
attcgttgatagggctaaagagatgaaagttgtagactgagatggatgaattat

SEQ ID NO: 47 - DNA, 55 bp, Arabidopsis thaliana
attcgttgatagggctaaagagatgaaaagttgtagactgagatggatgaattat

SEQ ID NO: 48 - DNA, 41 bp, Arabidopsis thaliana
attcgttgatgaaaagttgtagactgagatggatgaattat

SEQ ID NO: 49 - DNA, 58 bp, Arabidopsis thaliana
attcgttgatagggctaaagagattgggaaaagttgtagactgagatggatgaattat

SEQ ID NO: 50 - DNA, 34 bp, Arabidopsis thaliana
attcgttgattgtagactgagatggatgaattat

SEQ ID NO: 51 - DNA, 30 bp, Arabidopsis thaliana
attcgttgataactgagatggatgaattat

SEQ ID NO: 52 - DNA, 32 bp, Arabidopsis thaliana
attcgttgattagactgagatggatgaattat

SEQ ID NO: 53 - DNA, 36 bp, Arabidopsis thaliana
attcgttgatgttgtagactgagatggatgaattat

SEQ ID NO: 54 - DNA, 30 bp, Arabidopsis thaliana
attcgttgatgactgagatggatgaattat

SEQ ID NO: 55 - DNA, 35 bp, Arabidopsis thaliana
attcgttgatttgtagactgagatggatgaattat

SEQ ID NO: 56 - DNA, 36 bp, Arabidopsis thaliana
attcgttgatattgtagactgagatggatgaattat

SEQ ID NO: 57 - DNA, 38 bp, Arabidopsis thaliana
attcgttgataagttgtagactgagatggatgaattat

SEQ ID NO: 58 - DNA, 39 bp, Arabidopsis thaliana
attcgttgataaagttgtagactgagatggatgaattat

SEQ ID NO: 59 - DNA, 41 bp, Arabidopsis thaliana
attcgttgataaaaagttgtagactgagatggatgaattat

SEQ ID NO: 60 - DNA, 42 bp, Arabidopsis thaliana
attcgttgatggaaaagttgtagactgagatggatgaattat

SEQ ID NO: 61 - DNA, 45 bp, Arabidopsis thaliana
attcgttgatatgggaaaagttgtagactgagatggatgaattat

SEQ ID NO: 62 - DNA, 44 bp, Arabidopsis thaliana
attcgttgattgggaaaagttgtagactgagatggatgaattat

SEQ ID NO: 63 - DNA, 37 bp, Arabidopsis thaliana
attcgttgatagttgtagactgagatggatgaattat

SEQ ID NO: 64 - DNA, 31 bp, Arabidopsis thaliana
attcgttgatagactgagatggatgaattat

SEQ ID NO: 65 - DNA, 43 bp, Arabidopsis thaliana
attcgttgatgggaaaagttgtagactgagatggatgaattat

SEQ ID NO: 66 - DNA, 29 bp, Arabidopsis thaliana
attcgttgatactgagatggatgaattat

SEQ ID NO: 67 - DNA, 34 bp, Arabidopsis thaliana
attcgttgataggagactgagatggatgaattat

SEQ ID NO: 68 - DNA, 32 bp, Arabidopsis thaliana
attcgttgataggactgagatggatgaattat

SEQ ID NO: 69 - DNA, 16123 bp, Artificial Sequence
  vector 27057
  misc_feature (4)..(143): bNRB
  promoter (217)..(741): pr35S
  gene (767)..(2080): cGal4VP16GR
  terminator (2092)..(2734): tPsE9
  promoter (2943)..(2989): pr35Smin
  gene (3041)..(6913): cLbCas12a
  terminator (6921)..(7160): tPI2
  promoter (7184)..(8797): prGmSAMS
  misc_RNA (8847)..(8951): HH_AtGL1gRNA_HDV
  terminator (8959)..(9304): tAtUbq
  promoter (9339)..(9684): prCMP
  gene (9695)..(10489): cNpt2
  terminator (10518)..(10770): tNOS
  misc_feature (10855)..(10984)
  misc_feature (10985)..(16123): vector backbone
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagtttaaacaatggcagatccaatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctaggtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccaattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgacttgagctcttagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaataacctgcaggtcgaagcttgcatgccggtcgactctagaggatccgggtgacagccctccgacgggtgacagccctccgacgggtgacagccctccgaattctagaggatccgggtgacagccctccgacgggtgacagccctccgacgggtgacagccctccgaattcgagctcggtacccggggatctgtcgacctcgatcgagatcttcgcaagacccttcctctatataaggaagttcatttcatttggagaggacacgctgaagctagtcgactctagcctgcgatcgctaaaaagctagcatgccaaagaagaagagaaaggttggaggtggaggttctggaggtggaggttctggaggtggaggatctggaggtggaggttctggaggtggaggatctggaggtggaggtagtatgagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcaggaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaagcttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaaccctagcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctggacaaggtgaagatcgccatctccaacaaagagtggcttgagtacgctcagacctccgttaagcacggatctccaaagaagaagagaaaggtttctggaggatcttctggaggatcaccaaagaagaagagaaaagtttgacctgcaggaccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatataccggacttcgaattaactagctagtccaccctcacaaaaaaggttattgcctaattaatagtagggattgaagaagtatcaataatcaataagagggacatttggattgacacaaactattagtataaaaaaaaaatgtaaaactaaatggacaacaatctaagaaagcgagtatgtgttgtactcggaaaacataacataatgtgattttgattgtggaaactgaaaacaataacaattaagttttatttatctctttgcctaacaatttttttaattactttttttatagtttttatgatgaagataattaatattttttacaaatattatattttcctttttaccatctttgaagataattgcttttttttccaatttagacaaatatttgagtatcaatgatattcctttttaatccattattgcaatttaaaagaccatcaatgattacattttcaatcaaatgactttattagtcttcactttacatcgatttaaatcaaactaataattttgtatggactaaatctctgaacatttttatatttacaacatattttaacatttattaaataattagtatttaatactattagtagaataatgggagtagcaggagggaggcactgagagaatagagatggcatggaagtaagcaatcaagtcaaaatcagagttggccaaccccaaaggctgtagtaggtaagcatggcccattttagtttttacattcatctctcattttcacctcaacggttcagattcaatctgactccccgatctcagccgtggattcaaatgccacctcaggcacatgcaattccaaatggatgaacctaacccacaatctaatcttgttacttaggggcttttccgtcattaaatgacaccacctacccccttctccctataaatggcaactcaatcccccccttagaactcgcagcgcttgatttgaggccaggcaagccccactcaaccaccacacctctcctcgttcacgctacccctttctgctcttcttctacctttcaaggtactcttctttccctctgttgctgcaaccttctctttctttaagattgcctcaatttcggatcttgcacctctgggttgctttgctttgctttttcctctactgggttgatttctgtttccctaaaccggtttagacgaatgtgaacactacttcttttgtttaattactctggaatacgtgttaggctttcagatctagttgaaatcgtattgcacttttagggggagtttggatttctaataagaaattgaccttttgctgagaattggttcggtgattagagggtttccgtaaatttttgaagttttacatgcttgtatctgtttatttttgtttctcacatctattattgttaggtgaaggaaattatgtattgagagtctgtctgatactaaatataaacacctcaataggggctctaacactgattttatcatttgctgcttgtgtgtatggttaaagaaaggcaattgtgttttaattttctgcaagctttcgtttgctgaattttatgcatatattttcctcccttttgtgaacttcctttttgtagttctaattccatttttggtgtctgcagttttaaaagtataaagggatccaaattactgatgagtccgtgaggacgaaacgagtaagctcgtctaatttctactaagtgtagatccacatctctttagccctatcaaggccggcatggtcccagcctcctcgctggcgccggctgggcaacatgcttcggcatggcgaatgggacagctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaacttgaatgttcactactacggaccgctagtgcatgcactgactaattagctaactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaagacctaaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccacggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcattagcctgcaggcccgggttagtctaattagctaacggccaggatcgccgcgtgagcctttagcaactagctagagcgatcgccgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacagataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta